(19) United States
(12) Patent Application Publication
(10) Pub. No.: US 2004/0087512 A1
(43) Pub. Date: May 6, 2004

(54) In-situ formation of CLA

(76) Inventors: Beverly B. Teter, College Park, MD (US); Joseph Sampugna, University Park, MD (US); Richard A. Erdman, Columbia, MD (US); Liliana Piperova, Greenbelt, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/445,059

(22) Filed: May 27, 2003

Related U.S. Application Data
(63) Continuation of application No. 09/700,162, filed on Mar. 7, 2001, now abandoned.
Application No. 09/700,162 is a 371 of international application No. PCT/US99/10726, filed on May 12, 1999.
(60) Provisional application No. 60/085,199, filed on May 12, 1998.

(51) Int. Cl. A61K 31/202; A61K 31/70; A61K 35/78

(52) U.S. Cl. 514/23; 514/560; 424/750

(57) ABSTRACT

This invention relates to increasing levels of 18:1 trans fatty acids in production animals, the products of which may then be fed to mammals which in turn leads to the production of CLA from ingested 18:1 trans fatty acids; a method to screen diets using a mouse model to detect the effect on milk fat production and content; and a method and device for milking a mouse.

### FIELD OF THE INVENTION

This invention relates to increasing levels of 18:1 trails fatty acids in production animals, the products of which may then be fed to mammals which in turn leads to the production of CLA from ingested 18:1 trains fatty acids; a method to screen diets using a mouse model to detect the effects on milk fat production and content; and a method and device for milking a mouse.

### BACKGROUND OF THE INVENTION

Conjugated linoleic acid (CLA) is a mixture of positional and geometric isomers of linoleic acid—acids having lengths of 18 carbons with 2 conjugated double bonds. It is understood that conjugated linoleic acid is beneficial in suppressing the growth of breast cancer cells and tumeroigenesis (Wong), (Visonnes), (Cunningham), and in partitioning whole body energy (Pariza), It is also generally accepted that the 9c 11t isomer of linoleic acid is the most effective metabolically.

It is known that CLA results from the microbial biohydrogenation of polyunsaturated fatty acids (PUFA). It is also known that dairy products have been identified as the richest source of CLA's in the American diet. It is not known whether all the CLA appearing in milk or muscle tissue of ruminants is derived from the ruminal supply or is produced by the liver or the mammary gland from the 18:1 trans fatty acid (tFA) produced in the rumen and excreted in the milk or found in the muscle. However, it is known that the trans geometry of the bond is not formed in the mammalian system but must be of dietary origin—either by ruminal biohydrogenation or supplemented from partially hydrogenated oils.

As suggested above, trans isomers are not produced by the mammalian (including human) body. Thus, the current emphasis is to feed CLA's to animals or humans. Unfortunately, a diet supplement of free fatty acids or other compounds or triglycerides containing CLA, for instance a supplement of 9c 11t CLA is not always beneficial. Supplements currently available are mixtures known to be antimicrobial and, thus, their ingestion may be deleterious to digestive microbia. Studies have also shown that the eggs of laying birds failed to hatch and that tumor loads in mice decreased when such subjects were directly fed CLA. Thus, there is an indication that CLA's inhibit meiosis. In situ production of CLA's would prevent some of the reported side effects of CLA. In addition the in situ reaction could be directed to produce only those CLA Isomers deemed beneficial and provide consumers with the reported benefits of CLA consumption (i.e. 9c 11t or others). Furthermore, the acid conditions in the human stomach may lead to isomerzation of the 9c, 11t isomer when ingested but if precursors are fed and desaturated in situ only desired CLA's would be produced in the tissues.

In addition, the current attempts to provide CLA's of animal origin are difficult to control. Biohydrogenation may not stop at the CLA step and thus provide variable levels in food. The optimal dosages for maximum benefit are not known and high levels of consumption may have adverse as well as beneficial effects. Recent efforts to feed humans partially hydrogenated vegetable oils in order to increase serum CLA's has the disadvantage of producing isomers other than 9c 11t as well as the documented adverse effects of trans 18:1 and possibly as 18:1 isomers. Feeding 18:1 t11 enriched diets would obviate deleterious effects of other isomes.

### SUMMARY OF THE INVENTION

The inventors increase levels of the trans fatty acids in production animals, the products of which are then consumed by humans. In order to derive CLA benefits it is necessary for mammals to ingest fatty acid trans isomers such as the 18:1t-11 isomer. This isomer is the major isomer in dairy fat and one of the major isomers present in partially hydrogenated vegetable oils, but process dependent. The 18:1t-11 isomer is desaturated in any mammalian body by a common enzyme, delta-9 desaturase, at the n-9 position yielding 9c 11t CLA, —the CLA isomer reported to be most beneficial. Likewise, certain trams 18:1 and cis fatty acids can be desaturated by the animal liver or mammary gland at the delta-9 position. The trans 18:1 n-11 is one of the isomers capable of being desaturated in this manner. Providing the appropriate substrate, (i.e. t18:1 n-11, trans vaccinic acid) would allow the mammary gland to produce 9c 11t 18:2 by action of the delta-9 desaturase which is active in the gland during lactation, Likewise, in beef cattle as well as dairy cows, the liver as well as other tissues are able to produce CLA's trans-monoene substrates.

Thus, an object of the invention is to allow for an animal or person to produce their own CLA's in situ; such a process is under metabolic controls and the levels appropriate for the individual organism.

Another object of this invention is to alter rumen conditions to lead to variations, i.e., increases in the amounts of tFA (trans 18:1 and trans 18:2 fatty acids) including CLA's produced by the production animal.

Another object of the invention is to design diets which provide the appropriate oil substrates and rumen environment to optimize the production of tFA.

Another object of the invention is to provide a method for increasing the production of cis, trans conjugated acids in a mammal comprising, feeding to a mammal the food products of production animals fed diets high in carbohydrates, said food products containing increased levels of 18:1 trans-11 acid as a result of consuming diets high in carbohydrates and fat.

Another object is to provide a feed supplement or feed composition that prevents lactation failure in mammals.

Another object is to provide a small animal model wherein a specific diet produced is fed to the small animal and meat and milk products are analyzed to predict milk and meat composition of larger production animals.

### BRIEF DESCRIPTION OF THE DRAWINGS

FIG. 1 shows ruminal biohydration of linoleic acid (18:2 n-6)

FIG. 2 shows ruminal biohydration of linoleic acid (18:3 n-3)

FIG. 3a and 3b show a device used to milk mice.

### DETAILED DESCRIPTION OF THE INVENTION

Definitions

As used herein a production animal is defined as an animal such as beef and dairy cows, swine, lamb, chicken, duck, goose, emu, ostrich and fish.

FCM—fat corrected milk

GRAS—Generally recognized as safe.

DIM—days in milk

DIN—dry matter intake

DM—dry matter

FAME—fatty acid methyl ester

tFA—trans fatty acid whether containing a single double bond or multiple double bonds, conjugated or unconjugated.

CLA—a mixture of positional and geometric isomers of linoleic acid—acids having carbon lengths of 18 plus two double bonds which are conjugated.

PHVS—partially hydrogenated vegetable shortening

PHVO—partially hydrogenated vegetable oil

PUFA—polyunsaturated fatty acids

HOSO—high oleic sunflower oil

HLSO—high linoleic sunflower oil

FA—fatty acid

VFA—volatile fatty acid

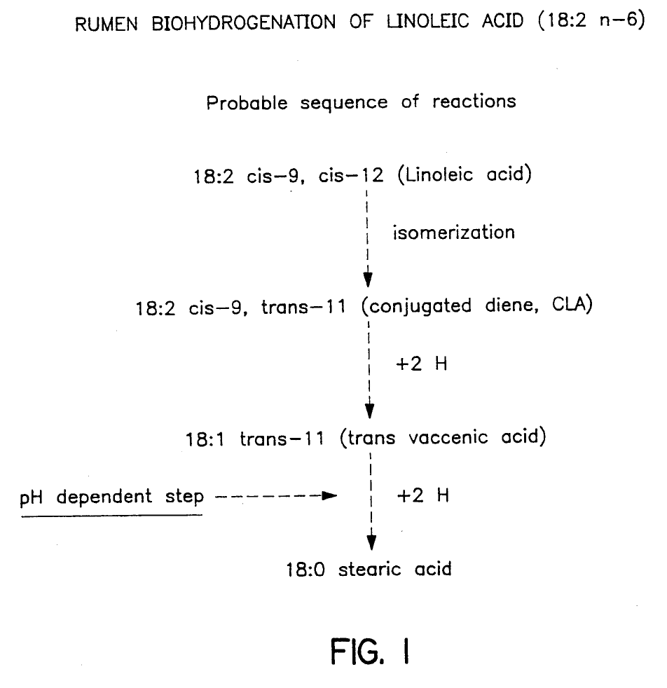

In the rumen the production of tFA is a several step process which depends on the fat and fatty acid substrates present in the rumen. Ruminants fed high amounts of linoleic acid i.e., diets high in corn oil, will biohydrogenate such acid as shown in FIG. 1. The last step in the process is pH dependent. Thus, allowing for the accumulation of 18:1 trans 11 (trans vaccinic acid) by providing linoleic acid containing feeds as a substrate and/or lowering the pH of the rumen prior to the conversion of trams vaccinic acid to stearic acid, allows for the accumulation of the substrate for production of CLA's found in the food products of production animals. The 18:t 11 isomer is GRAS since it has been present in dairy products for years.

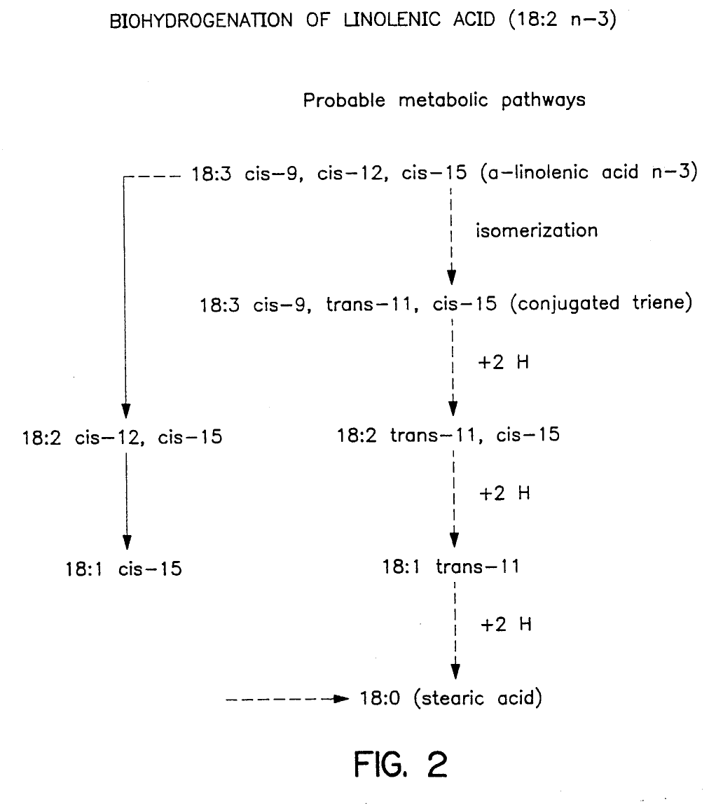

Ruminants fed high amounts of linoleic acid, i.e., diets high in oils such as soy oil, will biohydrogenate such acid as shown in FIG. 2. Again, the last step in the process is pH dependent. Thus, allowing for the accumulation of 18:1 trans 11 (trans vaccinic acid) by providing linoleic acid containing feeds as a substrate and/or lowering the pH of the rumen prior to the conversion of trans vaccinic acid to stearic acid allows for the accumulation of the substrate for production of CLA's and CLA's themselves found in the food products (milk, cheese and meat) of production animals.

Thus, decreased pH leads to more tFA formation. Increased levels of polyunsaturated fatty acids (PUFA) 18:2 and 18:3 leads to more tFA production. A feed of all forage produces mostly 18:1 t11n-7 in the rumen (Katz and Keeney). Appropriate rumen conditions and feed can alter the level tFA formed and the isomer distribution By controlling rumen conditions its is possible to direct the levels and isomer distribution of tFA produced.

In the case of non-ruminants the diet of the animal contains the substrate (i.e. 18:1t 11n-7) which would be absorbed in the digestive tract and provide the substrate to the tissues.

### EXAMPLE 1

The object of this example is to show the effects of dietary fat source on duodenal flow, apparent absorption, and milk fat incorporation of trans 18:1 fatty acids. (See Kalscheur et. al, “Effect of Fat Source on Duodenal Flow of Trans-C_{18:1} Fatty Acids and Milk Fat Production in Dairy Cows” J. Dairy Science 80:2115-2126 (1997) herein incorporated by reference)

Materials And Methods

Cows, Experimental Design, and Treatments

Four multiparous Holstein cows averaging 213 DIM at the beginning of the experiment were fitted with ruminal and duodenal (T-type gutter channel) cannulas. Duodenal cannulas were inserted proximal to the common bile and pancreatic duct, approximately 10 cm distal to the pylorus. Treatments, applied in a 4×4 Latin square design, consisted of either 0% supplemental fat the control diet 3.7% supplemental HOSO (Trisun 100A high oleic sunflower oil; SVO Enterprises, Eastlake, Ohio), 3.7% supplemental HLSO (Food grade sunflower oil; Columbus Foods Co., Chicago, Ill.), or 3.7% supplemental PHVS (Heavy duty vegetable frying shortening; Bunge Foods, Bradley, Ill.). Supplemental fat was substituted for corn in the basal diet. The FA compositions of the fat supplements are presented in Table 1.

TABLE 1
Long-chain fatty acid (FA) composition of high oleic
sunflower oil (HOSO), high linoleic sunflower oil (HLSO),
and partially hydrogenated vegetable shortening (PHVS).

| | HOSO¹ | HLSO² | PHVS³ |
|---|---|---|---|
| FA | (g/100 g of fat) | | |
| C_{14:0} | ND⁴ | ND | 0.08 |
| C_{16:0} | 4.15 | 6.58 | 10.14 |
| C_{16:1} | 0.05 | 0.02 | ND |
| C_{17:0} | 0.01 | 0.01 | 0.11 |
| C_{18:0} | 4.13 | 4.61 | 12.57 |
| trans-C_{18:1} | ND | 0.59 | 43.23 |
| cis-C_{18:1} | 77.91 | 20.16 | 28.83 |
| C_{18:2} | 11.09 | 65.18 | 0.63 |
| C_{18:3} | 0.02 | 1.25 | 0.21 |
| C_{≧20:n}⁵ | 2.62 | 1.60 | 3.70 |

¹Trisun 100A high oleic sunflower oil (SVO Enterprises, Eastlake, OH).
²Food grade sunflower oil (Columbus Foods Co., Chicago, IL).
³Heavy duty vegetable frying shortening (Burge Foods, Bradley, IL).
⁴Not detected.
⁵Includes saturated and unsaturated FA.

The diets were formulated to meet NRC (16) guidelines for milk production at 40 kg/day with 3.8% fat. Ingredient and nutrient compositions of the diets are shown in Table 2. The forage portion of each diet consisted of 60% corn silage and 40% alfalfa haylage. The DM percentage of forage and concentrate was determined weekly and the total mixed diets were adjusted accordingly to maintain a constant forage to concentrate ratio on a DM basis. The FA composition of the dietary ingredients and the calculated FA composition of the total mixed diets are presented in Table 3.

TABLE 2
Ingredients and chemical composition of total mixed diets
for cows fed the control diets supplemented with high oleic
sunflower oil (HOSO), high linoleic sunflower oil (HLSO),
or partially hydrogenated vegetable shortening (PHVS).

| | Diet | | | |
|---|---|---|---|---|
| | Control | HOSO | HLSO | PHVS |
| Composition | (% of DM) | | | |
| Ingredient | | | | |
| Corn silage | 36.0 | 36.0 | 36.0 | 36.0 |
| Alfalfa haylage | 24.0 | 24.0 | 24.0 | 24.0 |
| Ground corn | 20.5 | 17.0 | 17.0 | 17.0 |
| Soyplus ®¹ | 17.0 | 16.9 | 16.9 | 16.9 |
| Dicalcium phosphate | 0.9 | 0.9 | 0.9 | 0.9 |
| Limestone | 0.5 | 0.5 | 0.5 | 0.5 |
| NaCl | 0.4 | 0.4 | 0.4 | 0.4 |
| Dynamate ®² | 0.4 | 0.4 | 0.4 | 0.4 |
| Trace mineral and vitamin mix³ | 0.2 | 0.2 | 0.2 | 0.2 |
| Oleic sunflower oil | — | 3.7 | — | — |
| Linoleic sunflower oil | — | — | 3.7 | — |
| Vegetable shortening | — | — | — | 3.7 |
| Chemical | | | | |
| DM, % | 46.3 | 46.5 | 46.5 | 46.5 |
| OM | 92.2 | 92.1 | 92.2 | 92.1 |
| CP | 20.2 | 19.8 | 19.8 | 20.1 |
| NDF | 35.4 | 34.9 | 35.0 | 34.6 |
| ADF | 19.6 | 19.4 | 22.3 | 19.2 |
| Ca | 0.94 | 0.98 | 0.96 | 0.96 |
| P | 0.57 | 0.56 | 0.54 | 0.56 |
| Mg | 0.25 | 0.24 | 0.24 | 0.24 |
| K | 2.01 | 2.01 | 1.99 | 2.02 |
| Na | 0.20 | 0.18 | 0.18 | 0.19 |
| NE_{L},⁴ Mcal/kg of DM | 1.66 | 1.80 | 1.80 | 1.80 |

¹Soyplus/West Central (Ralston, IA).
²Pitman-Moore, Inc. (Chicago, IL); guaranteed analysis: 22% S, 18% K, and 11% Mg.
³Formulated to provide (per kilogram of dietary DM) 3000 IU of vitamin A, 500 IU of vitamin D, 10 IU of vitamin E, 50 mg of Fe, 40 mg of Zn, 40 mg of Mn, 10 mg of Cu, 0.6 mg of l, and 0.3 mg of Sc.
⁴Calculated from NRC (16).

TABLE 3
Fatty acid (FA) composition of concentrates, forages, and total
mixed diets for cows fed the control diet or diets supplemented
with high oleic sunflower oil (HOSO), high linoleic sunflower oil
(HLSO), or partially hydrogenated vegetable shortening (PHVS).

| | Concentrate mix | | | | Corn | Alfalfa | Diet | | | |
|---|---|---|---|---|---|---|---|---|---|---|
| | Control | HOSO | HLSO | PHVS | silage | haylage | Control | HOSO | HLSO | PHVS |
| FA | (% of DM) | | | | | | | | | |
| C_{16:0} | 0.636 | 1.024 | 1.245 | 1.388 | 0.489 | 0.513 | 0.552 | 0.705 | 0.791 | 0.847 |
| C_{16:1} | 0.002 | 0.002 | 0.003 | 0.002 | 0.009 | 0.045 | 0.015 | 0.015 | 0.015 | 0.015 |
| C_{18:0} | 0.151 | 0.519 | 0.573 | 1.120 | 0.075 | 0.095 | 0.110 | 0.254 | 0.275 | 0.490 |
| trans-C_{18:1} | ND¹ | 0.008 | 0.054 | 2.461 | 0.029 | ND | 0.014 | 0.014 | 0.031 | 0.975 |
| cis-C_{18:1} | 1.082 | 6.763 | 2.928 | 3.586 | 0.568 | 0.074 | 0.650 | 2.879 | 1.376 | 1.633 |
| C_{18:2} | 2.676 | 3.502 | 7.015 | 3.172 | 1.589 | 0.418 | 1.734 | 2.061 | 3.441 | 1.933 |
| C_{18:3} | 0.277 | 0.308 | 0.353 | 0.329 | 0.157 | 0.832 | 0.365 | 0.377 | 0.395 | 0.385 |
| Other² | 0.043 | 0.278 | 0.197 | 0.208 | 0.086 | 0.264 | 0.128 | 0.276 | 0.242 | 0.247 |
| Total | 4.867 | 12.406 | 12.368 | 12.266 | 3.002 | 2.241 | 3.565 | 6.581 | 6.566 | 6.525 |

¹Not detected
²Includes C_{14:0}, C_{17:0}, and C_{≧20:0}.

Experimental Procedure

Each experimental period consisted of 3 wk of which the first 16 d were used as an adjustment period. Cows were housed in individual tie stalls and were fed for ad libitum intake (allowing for 5 to 10% orts) once daily at 0800 h. Fats were blended into the concentrate every 10 or 11 d with a tumble mixer (Steiner Roll-A-Mix; Steiner Corp., Orrville, Ohio). At each feeding, dietary ingredients were thoroughly mixed. Feed intakes and orts were recorded daily. Samples of corn silage, alfalfa haylage, and concentrates fed to each cow and orts from each cow were collected on d 17 to 20 of each period, composted, oven-dried at 65° C, and ground through a Wiley mill (1-mm screen; Arthur H. Thomas, Philadelphia, Pa.) for later analysis. Milk production was recorded at each milking (0100 and 1230 h). Morning and evening milkings were sampled during the last 3 d (six consecutive milkings) of each period. Cows were exercised each morning from 0600 to 0800 h. Body weight was recorded weekly at 1100 h every Tuesday.

From d 11 to 21 of each period, Cr₂O₃ was mixed with the concentrate portion of the diet at aerate of approximately 20 g/d per cow based on previous DM intake to measure duodenal nutrient flow and to calculate total tract digestibility. Actual Cr₂O₃ intakes were determined by chemical analysis of the feeds and orts. Duodenal and fecal samples (250 ml) were collected every 2 h over a 24-h period. Duodenal samples were collected after discarding the first surge of digesta to help reduce bile contamination. Duodenal samples were frozen at −20° C, thawed, composited, and freeze-dried before being ground through a 1-mm screen.

Ruminal fluid samples for VFA and pH were collected every 2 h over a 12-h duration on d 21 of each experimental period beginning just before the morning feeding. Samples were collected in five distinct locations (10 ml of ruminal fluid per location) using a stainless steel sampling probe. Ruminal fluid pH was determined by glass electrode, acidified to pH<2 with 1 ml of 50% (vol/vol) H₂SO₄, and frozen for VFA analysis.

Ruminal samples for isolating bacteria were obtained on d 211 of each period at 0, 4, 8, and 12 h after the morning feeding. Whole ruminal contents, acquired from the anterior, dorsal, and midventral regions of the rumen, were squeezed through four layers of cheesecloth. Five hundred milliliters of the strained ruminal fluid were collected at each sampling, combined with 125 ml of a 0.9% NaCl and 37% formaldehyde solution to preserve ruminal bacteria, and refrigerated at 5° C until centrifugation to isolate bacteria. Remaining ruminal contents were returned to the cow. Composited samples were centrifugated at 500 (g for 15 min at 4° C. to remove feed particles and protozoa. The supernatant was centrifuged at 20,000 (g for 20 min at 4° C., washed with 0.9% NaCl, recentrifuged, and rinsed with distilled water to isolate the bacteria from the suspension. The remaining pellet was frozen, freeze-dried, and ground through a 1-mm screen for later analysis.

Analytical Procedures

Composited samples of alfalfa haylage, corn silage, concentrates, orts, duodenal digesta, and feces were analyzed for DM (100° C for 24 h), OM (1), and N by the micro-Kjeldahl procedure (1). Feed samples were additionally analyzed for NDF (25), ADF (10), Ca, P, Mg, K, and Na (Northeast DHIA Forage Analysis Laboratory, Ithaca, N.Y.). Long-chain FA compositions of feed, fecal, duodenal and milk samples were quantified by GLC as described by Gaynor et al. (9), except that feed, fecal, and duodenal methyl esters were purified through a silicic acid column (Bio-sil A 100-200 mesh; Bio-Rad Laboratories, Hercules, Calif.) to remove contaminants prior to GLC analysis. Individual milk samples were analyzed for fat, protein and lactose by Environmental Systems Services (College Park, Md.) using infrared analysis (Foss Milkoscan 104AB; Foss Food Technology Corp., Eden Prairie, Minn.). Ruminal VFA concentrations were determined by gas chromatography (Sigma 300; Perkin-Elmer Corp., Norwalk Conn.) using ethyl butyrate as an internal standard in a 2-m glass column packed with GP 15% SP-1220/1% H₃PO₄ on 100/120 Chromosorb®(Supelco Inc., Bellefonte, Pa.). Helium was the carrier gas and the injector, column, and detector temperatures were 200, 140, and 220° C., respectively.

Concentrates, orts, duodenal digesta, and feces were analyzed for Cr according to the procedure of Suzuki and Early (23). Duodenal and total tract flows of DM were calculated by dividing the amount of daily Cr intake by Cr concentration measured in duodenal and fecal samples. Nutrient flows were calculated by multiplying DM flow by the concentration of the nutrient in duodenal or fecal DM. Digestibilities, expressed as a percentage, were computed as the difference between nutrient intake and flows divided by intake. True ruminal DM digestibility was estimated by correcting duodenal DM flow for bacterial DM.

Bacterial pellet was analyzed for DM and N. Bacterial N flow to the duodenum was determined by purine analysis (29), as modified by Ushida et al. (24), of duodenal digesta and bacterial isolates. Ammonia N was measured by distillation of samples treated with MgO (1).

Results

Intakes And Digestibilities Of Nutrients

The FA content of the control diet was about 3.6% and was 6.6% for the fat-supplemented diets (Table 3). Total FA content measured in the fat-supplemented diets was about 0.7% (DM basis) lower than expected. Daily DM and OM intakes were numerically, but not significantly, lower for cows fed fat-supplemented diets by approximately 0.8 kg/d (Table 4). Apparent ruminal digestibility, true ruminal digestibility, and total tract digestibility of both DM and OM were not affected by treatment. Intake, duodenal flow, apparent ruminal digestibility, and total tract digestibility of NDF were not affected by treatment, but again tended to by lower for cows fed the fat-supplemented diets.

TABLE 4
Intake and digestibility of DM, OM, and NDF of cows fed the control diet
or diets supplemented with high oleic sunflower oil (HOSO), high linoleic
sunflower oil (HLSO), or partially hydrogenated vegetable shortening (PHVS).

| | | | | | | Contrast¹ | | |
|---|---|---|---|---|---|---|---|---|
| | Diet | | | | | C vs. F | O vs. L | L vs. S |
| | Control | HOSO | HLSO | PHVS | SEM² | P | | |
| DM | | | | | | | | |
| Intake, kg/d | 19.9 | 19.4 | 19.0 | 19.0 | 0.5 | 0.23 | 0.61 | 0.95 |
| Duodenal flow, kg/d | 15.5 | 17.0 | 15.9 | 15.5 | 0.7 | 0.43 | 0.31 | 0.66 |
| Apparent ruminal digestibility, % | 22.8 | 13.1 | 17.4 | 17.5 | 4.7 | 0.26 | 0.54 | 0.99 |
| True ruminal digestibility, % | 41.1 | 31.8 | 36.4 | 35.5 | 3.4 | 0.16 | 0.44 | 0.78 |
| Total tract digestibility, % | 66.4 | 62.4 | 64.1 | 60.7 | 2.9 | 0.28 | 0.71 | 0.44 |
| OM | | | | | | | | |
| Intake, kg/d | 18.4 | 17.9 | 17.5 | 17.5 | 0.5 | 0.22 | 0.64 | 0.91 |
| Duodenal flow, kg/d | 13.0 | 14.3 | 13.3 | 12.9 | 0.6 | 0.48 | 0.30 | 0.59 |
| Apparent ruminal digestibility, % | 29.6 | 20.6 | 25.0 | 25.4 | 4.4 | 0.28 | 0.51 | 0.95 |
| True ruminal digestibility, % | 46.3 | 37.7 | 42.2 | 41.7 | 3.2 | 0.18 | 0.41 | 0.81 |
| Total tract digestibility, % | 67.4 | 63.4 | 65.0 | 61.9 | 2.8 | 0.27 | 0.70 | 0.46 |
| NDF | | | | | | | | |
| Intake, kg/d | 7.0 | 6.8 | 6.6 | 6.6 | 0.2 | 0.15 | 0.65 | 0.82 |
| Duodenal flow, kg/d | 3.8 | 3.7 | 3.5 | 3.6 | 0.3 | 0.43 | 0.60 | 0.77 |
| Apparent ruminal digestibility, % | 45.9 | 45.7 | 48.4 | 44.9 | 4.8 | 0.94 | 0.70 | 0.62 |
| Total tract digestibility, % | 53.2 | 49.8 | 51.6 | 46.9 | 4.1 | 0.46 | 0.77 | 0.45 |

¹Contrasts:
C vs. F = control versus fat (HOSO), HLSO, and PHVS),
O vs. L = HOSO, and
L vs. S = HLSO versus PHVS.
²Four observations per treatment.

Intakes of individual and total FA are presented in Table 5. Total fatty acid intake of cows fed fat-supplemented diets increased by 533 g/d over that of cows fed the control diet because of the additional fat. Individual FA intakes were reflective of the FA composition of the feed ingredients (Table 3) as well as the FA composition of the supplemental fats (Table 1). Cows fed the fat-supplemented diets consumed greater quantities of C_{16:0}, C_{18:0}, trans-C_{18:1}, cis-C_{l8:1}, C_{18:2}, total C₁₈, and total FA. As expected, cows fed HOSO diets consumed greater cis-C_{18:1} and less C_{18:2} than did cows fed the PHVS diet. Fat supplementation increased trans-C_{18:1} FA intake because of the trans-C_{18:1} FA in the PHVS diet. Consumption of trans-C_{18:1} FA was negligible for cows fed the control, HOSO, and HLSO diets, but was 188 g/d for cows fed the diet supplemented with PHVS.

TABLE 5
Intake and flow of fatty acids (FA) to the duodenum of cows fed
the control diet or diets supplemented with high oleic sunflower
oil (HOSO), high linoleic sunflower oil (HLSO), or partially
hydrogenated vegetable shortening (PHVS).

| | | | | | | Contrast¹ | | |
|---|---|---|---|---|---|---|---|---|
| | Diet | | | | | C vs. F | O vs. L | L vs. S |
| FA | Control | HOSO | HLSO | PHVS | SEM² | P | | |
| Intake, g/d | | | | | | | | |
| C_{16:0} | 112 | 139 | 154 | 160 | 5 | 0.001 | 0.06 | 0.40 |
| C_{16:1} | 2.9 | 2.7 | 2.9 | 2.8 | 0.1 | 0.41 | 0.34 | 0.61 |
| C_{18:0} | 22 | 50 | 54 | 93 | 3 | 0.001 | 0.34 | 0.001 |
| trans-C_{18:1} | 2.0 | 2.6 | 6.4 | 187.9 | 7.1 | 0.001 | 0.72 | 0.001 |
| cis-C_{18:1} | 133 | 571 | 269 | 309 | 18 | 0.001 | 0.001 | 0.16 |
| C_{18:2} | 353 | 408 | 664 | 360 | 25 | 0.006 | 0.001 | 0.001 |
| C_{18:3} | 74 | 74 | 76 | 72 | 3 | 0.99 | 0.67 | 0.44 |
| Total C₁₈ | 584 | 1105 | 1069 | 1022 | 39 | 0.001 | 0.53 | 0.43 |
| Total | 722.0 | 1287.0 | 1259 | 1219 | 41 | 0.001 | 0.65 | 0.51 |
| Duodenal flow, g/d | | | | | | | | |
| C_{16:0} | 162 | 194 | 187 | 202 | 10 | 0.02 | 0.61 | 0.29 |
| C_{16:1} | 4.5 | 6.1 | 5.4 | 5.9 | 0.5 | 0.07 | 0.40 | 0.60 |
| C_{18:0} | 457 | 744 | 680 | 636 | 30 | 0.001 | 0.18 | 0.34 |
| trans-C_{18:1} | 64 | 287 | 295 | 266 | 37 | 0.002 | 0.89 | 0.61 |
| cis-C_{18:1} | 74 | 106 | 83 | 108 | 17 | 0.24 | 0.336 | 0.33 |
| C_{18:2} | 111 | 134 | 129 | 101 | 9 | 0.337 | 0.71 | 0.07 |
| C_{18:3} | 15.2 | 12.9 | 14.1 | 14.4 | 1.1 | 0.33 | 0.49 | 0.85 |
| Total C₁₈ | 721 | 1284 | 1200 | 1126 | 46 | 0.001 | 0.25 | 0.30 |
| Total | 955 | 1553 | 1470 | 1410 | 59 | 0.001 | 0.36 | 0.49 |
| Biohydrogenation of | | | | | | | | |
| unsaturated FA, % | | | | | | | | |
| cis-C_{18:1} | 54.9 | 83.8 | 71.7 | 68.3 | 5.0 | 0.01 | 0.14 | 0.65 |
| C_{18:2} | 74.6 | 73.0 | 83.0 | 74.6 | 1.2 | 0.15 | 0.001 | 0.003 |
| C_{18:3} | 83.2 | 84.7 | 83.3 | 81.7 | 1.2 | 0.99 | 0.45 | 0.40 |
| Total C₁₈ | 71.1 | 79.5 | 80.1 | 72.7 | 1.6 | 0.01 | 0.80 | 0.02 |

¹Contrasts:
C vs. F = control versus fat (HOSO, HLSO, and PHVS),
O vs. L = HOSO versus HLSO, and
L vs. S = HLSO versus PHVS.
²Four observations per treatment.
³Includes cis-C_{18:1}, C_{18:2}, and C_{18:3}.

Flows of both individual and total FA to the duodenum were increased by the diets supplemented with fat (Table 5). Total FA flow to the duodenum for cows fed fat-supplemented diets increased 598, 515, and 455 g/d for HOSO, HLSO, and PHVS treatments, respectively, compared with cows fed the control diet. Cows fed the fat-supplernented diets had increased duodenal FA flows of C_{16:0}, C_{16:1}, C_{18:0}, trans-C_{18:1}, total C₁₈, and total FA compared with cows fed the control diet. Duodenal flows of C_{18:0}, trans-C_{18:1} FA, cis-C_{18:1}, C_{18:2} for cows receiving the HOSO and BLSO treatments were quite similar despite differences in intake of these FA. The flow of C_{18:2} was lower in cows fed the PHVS diet than in cows fed the LSO diet, which was a reflection of the differences in FA intakes of the corresponding treatments. The flow of trans-C_{18:1} FA to the duodenum was greatly increased for cows fed the fat-supplemented diets compared with the cows fed the control diet (283 vs. 64 g/d; P<0.002); however, there was no effect of source of fat supplement.

Ruminal biohydrogenation of unsaturated C₁₈ FA in the diet was estimated from changes in the duodenum and intake of C_{18:0}, C_{18:1}, C_{18:2}, C_{18:3}, of total C₁₈ FA (28)

Biohydrogenation (percentage)=100−[100×(UFA/TFA)/(IUFA/ITFA)] Where

UFA=duodenal flow of an individual unsaturated C₁₈ FA,

TFA=duodenal flow of total C₁₈ FA,

IUFA=intake of an individual unsaturated C₁₈ FA, and

ITFA=intake of total C₁₈ FA.

Generally, as the degree of unsaturation increased, the extent of biohydrogenation increased, regardless of dietary treatment (Table 5). The one exception occurred for cows fed the HOSO diet for which biohydrogenation of C_{18:2} was lower than that of cis-C_{18:1}. Biohydrogenation of cis-C_{18:1} and total C₁₈ FA increased in cows fed the fat-supplemented diets compared with that in cows fed the control diet. Biohydrogenation of C_{18:2} was greater for cows consuming the HLSO diet than in cows consuming the HOSO or PHVS diet. Biohydrogenation of C_{18:3} was not affected by treatment. Cows fed the HLSO diet had greater biohydrogenation of total C₁₈ FA than did cows fed the PHVS diet.

The extent of biohydrogenation of unsaturated C₁₈ FA was also reflected by changes in the flow of C_{18:0} and trans-C_{18:1} FA to the duodenum. Flows of C_{18:0} increased in cows fed the fat-supplemented diets as expected because of biohydrogenation of the supplemented fat in the diet. For cows fed the control diet, duodenal flow of C_{18:0} was 63.3% of the total C₁₈ FA flow. Conversely, HOSO, HLSO, and PHVS treatments resulted in C_{18:0} proportions of 58.5, 57.7, and 56.3% of the total C₁₈ FA duodenal flow, respectively (P<0.008; contrast not shown). These data indicate that the proportion of C_{18:0} of total C₁₈ FA is decreased as the major en product of biohydrogenation when unsaturated FA are fed. The flow of trans-C_{18:1} FA, the product of incomplete biohydrogenation, was greatly increased for cows fed all fat-supplemented diets regardless of source. As a proportion of total C₁₈ FA flow to the duodenum, trans-C_{18:1} FA was increased from 9% of the total C₁₈ FA flow from cows fed the control diet to 23.5% of the total C₁₈ FA flow from cows fed the fat-supplemented diets. This result demonstrated that incomplete biohydrogenation occurred to a greater extent in cows fed fat-supplemented diets from which PUFA were supplied. In contrast, the increased flow of trans-C_{18:1} FA measured in cows fed the PHVS diet versus that in cows fed the HOSO or HLSO diet was probably due to dietary intake rather than incomplete biohydrogenation.

Fecal FA (grams/day) did not differ for C_{16:0}, C_{16:1}, cis-C_{18:1}, and C_{18:2} among treatments (Table 6). Fecal C_{18:0}, trans-C_{18:1}, total C₁₈, and total FA flows were greater in cows fed the fat supplemented diets, which corresponded with greater duodenal flows of FA. Across fat treatments, cows fed PHVS diets had greater fecal trans-C_{18:1} FA (P<0.09) than did cows fed HLSO diets.

TABLE 6
Fecal fatty acids and total apparent digestibility of fatty
acids by cows fed the control diet or diets supplemented with
high oleic sunflower oil (HOSO), high linoleic sunflower oil
(HLSO), or partially hydrogenated vegetable shortening (PHVS).

| | | | | | | Contrast¹ | | |
|---|---|---|---|---|---|---|---|---|
| | Diet | | | | | C vs. F | O vs. L | L vs. S |
| FA | Control | HOSO | HLSO | PHVS | SEM² | P | | |
| Focal flow, g/d | | | | | | | | |
| C_{16:0} | 40 | 48 | 50 | 56 | 6 | 0.14 | 0.80 | 0.52 |
| C_{16:1} | 2.6 | 2.7 | 2.8 | 2.6 | 0.5 | 0.84 | 0.92 | 0.78 |
| C_{16:2} | 125 | 258 | 241 | 240 | 29 | 0.01 | 0.69 | 0.99 |
| trans-C_{18:1} | 14 | 41 | 33 | 47 | 5 | 0.003 | 0.31 | 0.09 |
| cis-C_{18:1} | 18 | 18 | 23 | 19 | 3 | 0.61 | 0.33 | 0.37 |
| C_{18:2} | 20 | 27 | 19 | 226 | 3 | 0.37 | 0.18 | 0.23 |
| C_{18:3} | 2.5 | 2.9 | 1.7 | 2.7 | 0.4 | 0.91 | 0.06 | 0.11 |
| Total C₁₈ | 180 | 346 | 318 | 334 | 39 | 0.01 | 0.60 | 0.77 |
| Total | 249 | 426 | 399 | 421 | 48 | 0.02 | 0.67 | 0.74 |
| Total tract | | | | | | | | |
| digestibility, % | | | | | | | | |
| C_{16:0} | 63.4 | 65.1 | 67.6 | 65.0 | 5.30 | 0.70 | 0.75 | 0.75 |
| C_{16:1} | 3.9 | 1.1 | −13.8 | −2.5 | 22.8 | 0.75 | 0.66 | 0.74 |
| C_{16:2} | −477 | −414 | −350 | −159 | 102 | 0.20 | 0.67 | 0.23 |
| trans-C_{18:1} | −884 | −1599 | −870 | 75 | 376 | 0.85 | 0.22 | 0.13 |
| cis-C_{18:1} | 86.1 | 96.7 | 91.5 | 93.9 | 1.4 | 0.003 | 0.04 | 0.29 |
| C_{18:2} | 94.3 | 93.5 | 97.1 | 92.8 | 0.8 | 0.87 | 0.02 | 0.01 |
| C_{18:3} | 96.6 | 96.1 | 97.7 | 96.2 | 0.5 | 0.85 | 0.04 | 0.06 |
| Total C₁₈ | 68.7 | 68.4 | 70.2 | 67.2 | 5.5 | 0.99 | 0.82 | 0.71 |
| Total | 64.8 | 66.6 | 68.3 | 65.3 | 5.7 | 0.78 | 0.84 | 0.73 |

¹Contrasts:
C vs. F = control versus fat (HOSO, HLSO, and PHVS),
O vs. L = HOSO, and
L vs. S = HLSO versus PHVS.
²Four observations per treatment.

Apparent total tract digestibilities of C_{16:0}, C_{16:1}, C_{18:0}, trans-C_{18:1}, total C₁₈, and total FA were not affected by treatment (Table 6). The apparent digestibility of cis-C_{18:1} was increased (P<0.003) in cows fed fat-supplemented diets compared with that in cows fed the control diet. This result was most likely due to increases in ruminal biohydrogenation (Table 5). Apparent digestibility of cis-C_{18:1} in cows fed the LSO diet was lower (P<0.04) than that in cows fed the HOSO diet. Apparent digestibility of C_{18:2} and C_{18:3} was greater (P<0.04) for cows fed the LSO diet than for cows fed the HOSO or PHVS diet. Because of hydrogenation of unsaturated C₁₈ FA in the rumen, apparent digestibility of C_{18:0} was negative for all diets, resulting from a large quantity of C_{18:0} passing through the feces in comparison with consumption of C_{18:0}. The apparent digestibility of trans-C_{18:1} FA was negative for all diets that provided little dietary trans-C_{18:1} FA. For cows fed the PHVS diet, apparent digestibility of trans-C_{18:1} FA was positive, indicating the disappearance of trans-C_{18:1} FA from consumption of the the diet to excretion in feces.

Apparent postruminal digestibilities (data not shown) of total FA were 74.3, 72.2, 72.5, and 70.2% for cows fed the control, HOSO, HLSO, and PHVS diets, respectively, and did not differ among diets. There were few significant effects caused by source of dietary fat on apparent postruminal digestibilities of individual FA. Digestibility of cis-C_{18:1} decreased for cows fed HLSO diets compared with cows fed HOSO or PHVS diets (69.8% vs. 82.0%: P<0.04), and C_{18:2} decreased for cows fed PHVS diets compared with cows fed HLSO diets (74.4% vs. 83.5%; P<0.05). Although not significantly different, the fat-supplemented treatments tended to decrease apparent postruminal digestibility of C18:0 (63.9% vs. 75.4%), and increase digestibility of trans-C_{18:1} FA in the small intestine (85.2% vs. 78.8%) more than the control treatment.

Ruminal pH and VFA

Ruminal pH and VFA concentrations, averaged across sampling times, are shown in Table 7. Ruminal pH did not differ among treatments. Cows fed supplemental fat had lower total VFA concentrations in the rumen (76.8 vs. 83.2 mM; P<0.07) and a higher molar proportion of propionate decreased (19.0% vs. 19.8%; P<0.03), and butyrate increased (12.5% vs. 11.3%; P<0.09), in cows fed PHVS diets compared with cows fed HOSO diets There was no difference in ruminal VFA concentrations for cows fed HOSO diets compared with cows fed LSO diets.

TABLE 7
Ruminal pH and VFA concentration in cows fed the control diet or diets
supplemented with high oleic sunflower oil (HOSO), high linoleic sunflower
oil (HLSO), or partially hydrogenated vegetable shortening (PHVS).

| | | | | | | Contrast¹ | | |
|---|---|---|---|---|---|---|---|---|
| | Diet | | | | | C vs. F | O vs. L | L vs. S |
| | Control | HOSO | HLSO | PHVS | SEM² | P | | |
| pH | 6.60 | 6.51 | 6.62 | 6.48 | 0.09 | 0.58 | 0.36 | 0.25 |
| Total VFA, mM | 83.2 | 72.8 | 78.2 | 79.4 | 3.1 | 0.07 | 0.22 | 0.78 |
| VFA, mol/100 mol | | | | | | | | |
| Acetate | 64.3 | 64.3 | 64.3 | 64.0 | 0.6 | 0.86 | 0.97 | 0.65 |
| Propionate | 19.0 | 20.0 | 19.8 | 19.0 | 0.3 | 0.09 | 0.70 | 0.03 |
| Isobutyrate | 0.81 | 0.85 | 0.84 | 0.78 | 0.05 | 0.85 | 0.88 | 0.46 |
| Butyrate | 11.8 | 10.9 | 11.3 | 12.5 | 0.5 | 0.63 | 0.57 | 0.09 |
| Isovalerate | 1.71 | 1.63 | 1.60 | 1.62 | 0.07 | 0.28 | 0.84 | 0.87 |
| Valerate | 1.62 | 1.60 | 1.56 | 1.56 | 0.06 | 0.54 | 0.61 | 0.94 |

¹Contrasts:
C vs. F = control versus fat (HOSO, HLSO, and PHVS),
O vs. L = HOSO versus HLSO, and
L vs. S = HLSO vs. PHVS.
²Four cows per treatment; seven repeated measures.

N Metabolism

Intake of N was lower for cows fed fat-supplemented diets (660 vs. 619 g/d) because of lower DMI for those diets (Table 8). Duodenal flow of total N, ammonia N, NAN, bacterial N, and nonammonia, Nonbacterial N were not altered by treatment. Efficiency of bacterial protein synthesis (grams of bacterial N per kilogram of OM truly digested) was not affected by dietary fat. Fecal N and apparently digested N in the total tract did not differ across treatments.

TABLE 8
Digestion and microbial flow of N to the small intestine of cows
fed the control diet or diets supplemented with high oleic sunflower
oil (HOSO), high linoleic sunflower oil (HLSO), or partially
hydrogenated vegetable shortening (PHVS).

| | | | | | | Contrast¹ | | |
|---|---|---|---|---|---|---|---|---|
| | Diet | | | | | C vs. F | O vs. L | L vs. S |
| | Control | HOSO | HLSO | PHVS | SEM² | P | | |
| Intake, g/d | 660 | 628 | 617 | 612 | 11 | 0.02 | 0.53 | 0.74 |
| Duodenal flow, g/d | | | | | | | | |
| Total N | 697 | 705 | 678 | 684 | 33 | 0.84 | 0.60 | 0.91 |
| Ammonia N | 48 | 53 | 51 | 51 | 3 | 0.28 | 0.74 | 0.99 |
| NAN | 649 | 652 | 627 | 633 | 31 | 0.74 | 0.59 | 0.91 |
| Bacterial N | 287 | 269 | 273 | 251 | 21 | 0.38 | 0.90 | 0.48 |
| Nonammonia, nonbacterial N | 362 | 383 | 354 | 381 | 18 | 0.63 | 0.30 | 0.32 |
| Duodenal flow, g/100 g of N | | | | | | | | |
| Ammonia N | 6.8 | 7.4 | 7.6 | 7.5 | 0.3 | 0.07 | 0.67 | 0.78 |
| NAN | 93.2 | 92.6 | 92.4 | 92.5 | 0.3 | 0.07 | 0.67 | 0.78 |
| Bacterial N | 41.3 | 38.5 | 40.4 | 36.8 | 1.6 | 0.20 | 0.43 | 0.17 |
| Bacterial synthesis, g of | 35.1 | 42.8 | 39.1 | 36.5 | 6.0 | 0.55 | 0.67 | 0.77 |
| N/kg of OMTD³ | | | | | | | | |
| Fecal N, g/d | 207 | 211 | 201 | 213 | 16 | 0.93 | 0.67 | 0.60 |
| Total tract digestibility, % | 68.9 | 66.5 | 67.8 | 64.9 | 2.5 | 0.42 | 0.71 | 0.44 |

¹Contrasts:
C vs. F = control versus fal (HOSO, HLSO. and PHVS),
O vs L = HOSO, and
L vs. S = HLSO versus PHVS.
²Four observations per treatment.
³OM truly digested in the rumen.

Milk Production And Composition

Cows fed diets with supplemental fat had lower milk (P<0.06) and 3.5% FCM (P<0.02) production (Table 9). Milk fat percentage tended to decrease for cows fed fat-supplemented diets compared with cows fed the control diet (3.48% vs. 3.21%) but was not significant. Fat and protein production was reduced (P<0.03) for cows diets supplemented with fat. Source of fat supplementation did not alter milk fat and milk protein percentage or production.

TABLE 9
Body weight of cows and production and composition of milk from
cows fed the control diet or diets supplemented with high oleic sunflower
oil (HOSO), high linoleic sunflower oil (HLSO), or partially hydrogenated
vegetable shortening (PHVS).

| | | | | | | Contrast¹ | | |
|---|---|---|---|---|---|---|---|---|
| | Diet | | | | | C vs. F | O vs. L | L vs. S |
| | Control | HOSO | HLSO | PHVS | SEM² | P | | |
| BW, kg | 690 | 698 | 696 | 705 | 7 | 0.25 | 0.89 | 0.41 |
| Milk, kg/d | 26.1 | 23.9 | 25.3 | 22.9 | 0.8 | 0.06 | 0.26 | 0.07 |
| 3.5% FCM, kg/d | 26.1 | 21.8 | 24.2 | 22.3 | 0.9 | 0.02 | 0.11 | 0.20 |
| Milk components, % | | | | | | | | |
| Fat | 3.48 | 3.07 | 3.18 | 3.38 | 0.14 | 0.14 | 0.61 | 0.33 |
| Protein | 3.53 | 3.41 | 3.41 | 3.47 | 0.07 | 0.26 | 0.99 | 0.56 |
| Lactose | 4.75 | 4.67 | 4.73 | 4.54 | 0.10 | 0.40 | 0.64 | 0.21 |
| Production, kg/d | | | | | | | | |
| Fat | 0.91 | 0.71 | 0.81 | 0.76 | 0.04 | 0.03 | 0.14 | 0.46 |
| Protein | 0.92 | 0.81 | 0.85 | 0.78 | 0.03 | 0.03 | 0.33 | 0.18 |
| Lactose | 1.24 | 1.10 | 1.20 | 1.04 | 0.05 | 0.07 | 0.22 | 0.06 |

¹Contrasts:
C vs. F = control versus fat (HOSO, HLSO, and PHVS),
O vs. L = HOSO versus HLSO, and
L vs. S = HLSO versus PHVS.
²Four observations per treatment.

Concentrations and daily yield of individual FA in milk are shown in Table 10. Milk produced from cows fed diets that were supplemented with fat resulted in lower (P<0.01) weight percentages and yields of C_{14:0}, C_{15:0}, C_{16:0}, C_{17:0}, and C_{18:3}. Fat increased (P<0.01) the concentration of cis-C_{18:1} present in milk, but did not alter yield. The concentration of C_{18:2(n-6)} did not change, but the yield of C_{18:2(n-6)} decreased (P<0.004) for cows fed fat treatments compared with the yield for cows fed the control treatment. Cows fed fat-supplemented diets produced milk with a greater concentration (11.2% vs, 2.91%; P<0.004) and yield (81 vs. 26 g/d; P<0.001) of trans-C_{18:1} FA than did cows fed the control diet.

TABLE 10
Fatty acid (FA) concentration and production of milk from cows fed
the control diet or diets supplemented with high olele sunflower oil
(HOSO), high linoleic oil (HLSO or partially hydrogenated
vegetable shortening (PHVS).

| | Diet | | | | | Contrast¹ | | |
|---|---|---|---|---|---|---|---|---|
| FA | Control | HOSO | HLSO | PHVS | SEM2 | C vs. F | O vs. L | L vs. S |
| | (g/100 g of fat) | | | | | P | | |
| C_{14:0} | 13.0 | 8.9 | 10.0 | 9.6 | 0.5 | 0.001 | 0.23 | 0.63 |
| C_{15:0} | 1.21 | 0.89 | 0.90 | 0.94 | 0.03 | 0.001 | 0.94 | 0.41 |
| C_{16:0} | 32.2 | 22.0 | 3.7 | 26.3 | 0.7 | 0.001 | 0.10 | 0.03 |
| C_{16:1} | 2.2 | 1.8 | 1.6 | 2.3 | 0.2 | 0.17 | 0.49 | 0.04 |
| C_{17:0} | 0.59 | 0.47 | 0.47 | 0.49 | 0.02 | 0.003 | 0.90 | 0.44 |
| C_{18:0} | 12.0 | 13.7 | 13.8 | 12.2 | 0.6 | 0.12 | 0.83 | 0.09 |
| trans-C_{18:1} | 2.9 | 11.8 | 11.0 | 10.8 | 1.6 | 0.004 | 0.75 | 0.91 |
| cis-C_{18:1} | 22.4 | 28.5 | 24.8 | 25.9 | 1.0 | 0.01 | 0.03 | 0.44 |
| C_{18:2}³ | 1.0 | 2.1 | 2.2 | 1.8 | 0.2 | 0.002 | 0.77 | 0.11 |
| C_{18:2(0-6)} | 4.2 | 4.0 | 4.5 | 3.3 | 0.3 | 0.47 | 0.29 | 0.03 |
| C_{18:3} | 0.80 | 0.61 | 0.63 | 0.60 | 0.04 | 0.007 | 0.75 | 0.68 |
| C_{20:4} | 0.15 | 0.15 | 0.13 | 0.14 | 0.03 | 0.77 | 0.52 | 0.80 |
| | (g/d) | | | | | | | |
| C14:0 | 118 | 66 | 83 | 73 | 8 | 0.002 | 0.16 | 0.39 |
| C15:0 | 10.9 | 6.4 | 7.3 | 7.1 | 0.6 | 0.001 | 0.28 | 0.76 |
| C16:0 | 293 | 159 | 195 | 199 | 14 | 0.001 | 0.12 | 0.85 |
| C16:1 | 21 | 13 | 13 | 17 | 1.6 | 0.02 | 0.89 | 0.12 |
| C17:0 | 5.3 | 3.3 | 3.8 | 3.7 | 0.2 | 0.001 | 0.10 | 0.89 |
| C18:0 | 110 | 98 | 113 | 92 | 6 | 0.25 | 0.14 | 0.05 |
| trans-C_{18:1} | 26 | 79 | 81 | 82 | 7 | 0.001 | 0.85 | 0.90 |
| cis-C_{18:1} | 201 | 201 | 206 | 198 | 14 | 0.98 | 0.79 | 0.67 |
| C_{18:2} | 9 | 14 | 16 | 14 | 0.9 | 0.001 | 0.19 | 0.20 |
| C_{18:2(0-6)} | 37 | 27 | 35 | 25 | 1.6 | 0.004 | 0.01 | 0.005 |
| C_{18:3} | 7.3 | 4.3 | 5.2 | 4.8 | 0.3 | 0.001 | 0.09 | 0.38 |
| C_{20:4} | 1.2 | 1.0 | 1.1 | 1.0 | 0.3 | 0.54 | 0.95 | 0.87 |

¹Contrasts:
C vs. F = control versus fat (HOSO, HLSO, and PHVS),
O vs. L = HOSO versus HLSO, and
L vs. S = HLSO versus PHVS.
²Four observations per treatment.
³Isomers.

Because of the source of the fat treatment, cows fed the HOSO diet produced milk with greater concentrations of cis-C_{18:1} (P<0.03), but lower concentrations of C_{16:0} (P<0.10), than did cows fed the HLSO diet. Cows fed the HOSO diet also produced milk with lower daily yields of C_{18:2(n-6)} (P<0.01) and C_{18:3} (P<0.09) than did cows fed the LSO diet primarily because of decreased milk production. Comparing milk composition from cows fed HLSO and PHVS diets, cows fed the HLSO diet had lower concentrations of C_{16:0} and C_{16:1} in milk but greater concentrations and yields of C_{18:0} and C_{18:2(n-6)}.

Discussion

The net balance of total FA flow from intake to duodenal passage appears to vary with no known association with the characteristics of a specific fat, such as source, degree of unsaturation, or extent of ruminal protection (7). Although cows fed diets that were not supplemented with fat commonly have net increases of FA across the rumen (7, 15), cows fed different types of fats increase, decrease, or do not change the flow of FA to the duodenum (7, 15, 27, 28). Net increases of total FA across the rumen in the current study were 233, 266, 211, and 191 g/d higher than FA intake for cows fed control, HOSO, HLSO, and PHVS treatments, respectively. The net increase of FA could be accounted for from bacterial de novo lipid synthesis in the rumen (7, 11, 26) and possibly, bile contamination of duodenal digesta or other endogenous sources (17, 26).

Flow of trans-C_{18:1} FA into the small intestine compared with dietary intake increased for cows fed diets containing fat treatments; there was no effect of source. Duodenal flow of trans-C_{18:1} FA for cows fed the HLSO and PHVS diets was four times greater than that for cows fed the control diet. Increases in the flow of trans-C_{18:1} FA to the duodenum resulted from incomplete biohydrogenation for cows fed HLSO diets. For cows fed the PHVS diets, dietary consumption of trans-C_{18:1} FA was responsible for increases in duodenal flow. Unexpectantly, duodenal flow of trans-C_{18:1} for cows fed HOSO diets was comparable with flows in cows fed HLSO and PHVS diets, which might have been due to sufficient PUFA that were available for biohydrogenation even in the HOSO diet. It generally is assumed that cis-C_{18:1} is converted directly to stearic acid and that trans-C_{18:1} FA is not an intermediate in this process (11, 12). In agreement with previous research, Wonsil et al. (27) found duodenal trans-C_{18:1} FA flows for cows fed diets supplemented with menhaden fish oil or soybean oil to be four times greater than duodenal flows for cows fed the control or hydrogenated tallow diets. Increased flows for trans-C_{18:1} FA were attributed to incomplete hydrogenation of PUFA (27). Ruminal biohydrogenation of C_{18:2} was increased in cows fed the HLSO diet. Possibly this was due to increased accessibility of C_{18:2} to rumen microorganisms of the lipids in the oil portion of the diet than of lipids contained in cellular structures of concentrates and forages as suggested by Ferlay et al. (8). Similarly, biohydrogenation of cis-C_{18:1} in cows fed the HOSO diet was also higher than that in cows fed the other dietary treatments. Biohydrogenation of the C₁₈ unsaturated FA for cows fed the control diet followed a similar pattern compared with the control diets reported in previous research (13, 28).

The extent to which dietary unsaturated FA are biohydrogenated depends on environmental conditions in the rumen. Factors such as the FA composition of the diet, species of the ruminal bacteria, and pH of the ruminal fluid may influence how unsaturated FA are biohydrogenated. Research completed in vitro (Example 2) and in vivo (13) has suggested that low ruminal pH may interfere with the ruminal biohydrogenation pathway, reducing the conversion of trans-C_{18:1} FA to stearic acid in the final step of biohydrogenation, and consequently, causing an accumulation of trans-C_{18:1} FA in the ruminal fluid. This factor was not present in the current example because ruminal pH ranged from 6.48 to 6.62 and did not differ among dietary treatments. In this experiment trans-C_{18:1} FA accumulated because of the large amount of dietary PUFA that was available as substrate. Therefore, ruminal pH and substrate availability are two important factors that affect ruminal accumulation of trans-C_{18:1} FA.

Milk fat percentage was numerically decreased for cows fed HOSO and HLSO diets; however, no depression was observed for cows fed PHVS diets. In previous experiments, milk fat depression resulted in cows fed soybean oil (3), whole sunflower seeds (5), Ca salts of FA (6), and menhaden fish oil (27), all sources of fat that are high in PUFA. Milk fat depression has also been observed in cows fed sources of dietary trans-C_{18:1} FA, including hydrogenated vegetable oil (22, 27), ruminally protected hydrogenated soybean oil (2), and abomasally infused vegetable shortening (9, 18).

Milk fat from cows fed the PHVS diet contained high levels of trans-C_{18:1} FA in the milk and total milk fat production was reduced, but milk fat percentage was not affected. A recent study by Chouinard et al. (6) compared Ca salts of canola oil, soybean oil, and linseed oil supplemented at the rate of 4% of the total diet to dairy cows. All three fat treatments resulted in an increased percentage of trans-C_{18:1} FA in milk and reduced milk fat percentage; however, the greatest increase in the percentage of trans-C_{18:1} FA in milk came from cows fed the Ca salts of soybean oil (10.75 percentage units) followed by linseed oil (8.34 percentage units) and canola oil (6.58 percentage units), whereas the largest reduction in milk fat percentage was from cows fed the Ca salts of canola oil (1.38 percentage units) followed by soybean oil (1.07 percentage units) and linseed oil (0.49 percentage units). These studies suggest that although increased levels of trans-C_{18:1} FA in the milk are always seen during milk fat depression there is not a constant relationship between trans-C_{18:1} FA in milk and the degree of milk fat depression. Commercially hydrogenated vegetable fats contain a broad distribution of positional trans-C_{18:1} FA isomers from n-2 to n-12, the largest proportions of which are n-7, n-8, and n-9 isomers (19). Ruminal biohydration of unsaturated FA also results in numerous positional isomers of trans-C_{18:1} FA, but the predominant trans-C_{18:1} FA isomer is the n-7 isomer, vaccenic acid (4, 14). Although vaccenic acid was also the dominant positional isomer of trans-C_{18:1} FA throughout the blood plasma and into milk fat in the goat (4), it is possible that other positional isomers may be causative factor in reducing milk fat production. (See example 5)

In summary, from the above, duodenal flow of trans-C_{18:1} FA was increased for cows fed all supplemental fat diets. Increased flow of trans-C_{18:1} FA from cows fed HOSO and HLSO diets resulted from incomplete biohydration in the rumen, but the increased flow of trans-C_{18:1} FA from cows fed the PHVS diet was most likely from the diet. Trans-C_{18:1} FA were subsequently absorbed in the small intestine and incorporated into milk fat at equal amounts regardless of the source of the supplemental fat. Fat supplementation reduced mill fat production regardless of source. Cows fed HOSO and HLSO diets produced a lower milk fat percentage, but cows fed PHVS diets did not. In conclusion, trans-C18:1 FA are present during milk fat depression caused by dietary fat addition, but increased amounts of trans-C_{18:1} FA incorporated into milk fat does not necessarily translate into a proportional reduction in milk fat

### EXAMPLE 2

The object of this study was to show the effects of the amounts of dietary concentrate and buffer addition on duodenal flow, apparent adsorption, and milk fat incorporation of transa-C_{18:1} fatty acids. (See Kalscheur et. al, “Effect of Fat Source on Duodenal Flow of Trans-C_{18:1} Fatty Acids and Milk Fat Production in Dairy Cows” J. Dairy Science 80:2115-2126 (1997) herein incorporated by reference)

Materials And Methods

Cows, Experimental Design, And Treatments

Four multiparous Holstein cows averaging 131 DIM at the beginning of the experiment were fitted with ruminal and duodenal (T-type gutter channel) cannulas. Duodenal cannulas (plasticol) were inserted proximal to the common bile and pancreatic duct, approximately 10 cm distal to the pylorus. Dietary treatments consisted of two levels of forage, high (HF; 60%, DM basis) and low (LF; 25%, DM basis) and two levels of buffer, no buffer and 2% buffer. Buffer treatment consisted of 1.5% NaHCO₃ and 0.5% MgO supplemented for corn in the total mixed diet (DM basis). Treatments were applied in a 2×2 factorial arrangement within a 4×4 Lain square design.

Ingredient and nutrient compositions of the diets are presented in Table 11. The forage portion of each diet consisted of 60% corn silage and 40% alfalfa haylage. The diets were formulated to meet NRC (16) guidelines for milk production at 40 kg with 3.8% fat for all nutrients except fiber in the LF diet. The DM percentage of forage and concentrate was determined weekly, and the total mixed diets were adjusted accordingly to maintain a constant forage to concentrate ratio on a DM basis. The FA composition of the dietary ingredients and the calculated FA composition of the total mixed diets are presented in Table 12.

TABLE 11
Ingredient and chemical composition of total mixed
diets for cows fed diets that contained high (HF) and low
(LF) proportions of forage with (B) or without buffer (NB).

| | HF | | LF | |
|---|---|---|---|---|
| | NB | B | NB | B |
| Composition | (% of DM) | | | |
| Corn silage | 36.0 | 36.0 | 15.0 | 15.0 |
| Alfalfa haylage | 24.0 | 24.0 | 10.0 | 10.0 |
| Ground corn | 20.5 | 18.7 | 51.9 | 49.9 |
| Soyplus ®¹ | 17.0 | 16.9 | 8.8 | 8.8 |
| Soybean meal (48% CP) | — | — | 11.0 | 11.0 |
| Dicalcium phosphate | 0.9 | 0.9 | 0.8 | 0.8 |
| Limestone | 0.5 | 0.5 | 1.2 | 1.2 |
| NaCl | 0.4 | 0.4 | 0.4 | 0.4 |
| Dynamate ®² | 0.4 | 0.4 | 0.6 | 0.6 |
| KCl | — | — | 0.1 | 0.1 |
| Trace mineral and vitamin mix³ | 0.2 | 0.2 | 0.2 | 0.2 |
| MgO | — | 0.5 | — | 0.5 |
| NaHCO₃ | — | 1.5 | — | 1.5 |
| Chemical | | | | |
| DM % | 44.0 | 44.0 | 61.7 | 61.7 |
| OM | 92.1 | 91.1 | 92.6 | 91.3 |
| CP | 18.9 | 18.3 | 19.0 | 19.2 |
| NDF | 35.3 | 34.8 | 22.0 | 22.1 |
| ADF | 20.1 | 19.8 | 10.3 | 10.4 |
| Ca | 0.94 | 0.94 | 0.96 | 0.97 |
| P | 0.55 | 0.55 | 0.57 | 0.61 |
| Mg | 0.26 | 0.49 | 0.27 | 0.49 |
| K | 2.05 | 2.01 | 1.51 | 1.52 |
| Na | 0.18 | 0.56 | 0.25 | 0.60 |
| NE_{L},⁴ Mcal/kg of DM | 1.66 | 1.62 | 1.80 | 1.76 |

¹Soyplus/West Central (Ralston, IA).
²Pitman-Moore Inc. (Chicago, IL); guaranteed analysis: 22% S, 18% K, and 11% Mg.
³Formulated to provide (per kilogram of dietary DM) 3000 IU of vitamin A, 500 IU of vitamin D, 10 IU of vitamin E, 50 mg of Fe, 40 mg of Zn, 40 mg of Mn, 10 mg of Cu, 0.6 mg of l, and 0.3 mg of Sc.
⁴Calculated from NRC (16).

TABLE 12
Fatty acid (FA) composition of concentrates, forages, and
total mixed diets for cows fed diets that contained high
(HF) and low (LF) proportions of forage with (B) or without buffer (NB).

| | Concentrate mix | | | | | | Diet | | | |
|---|---|---|---|---|---|---|---|---|---|---|
| | HF | | LF | | Corn | Alfalfa | HF | | LF | |
| | NB | B | NB | B | silage | haylage | NB | B | NB | B |
| FA | (% of DM) | | | | | | | | | |
| C_{16:0} | 0.755 | 0.784 | 0.669 | 0.698 | 0.573 | 0.606 | 0.606 | 0.617 | 0.626 | 0.647 |
| C_{16:1} | 0.006 | 0.002 | 0.001 | 0.004 | 0.015 | 0.039 | 0.017 | 0.016 | 0.007 | 0.010 |
| C_{18:0} | 0.200 | 0.254 | 0.130 | 0.180 | 0.067 | 0.067 | 0.128 | 0.168 | 0.117 | 0.153 |
| trans-C_{18:1} | ND¹ | ND | ND | ND | 0.004 | ND | 0.001 | 0.001 | 0.001 | 0.001 |
| cis-C_{18:1} | 1.203 | 1.207 | 1.022 | 1.078 | 0.523 | 0.078 | 0.684 | 0.686 | 0.841 | 0.880 |
| C_{18:2} | 3.074 | 2.941 | 2.493 | 2.516 | 1.281 | 0.431 | 1.784 | 1.732 | 2.078 | 2.092 |
| C_{18:3} | 0.368 | 0.345 | 0.186 | 0.182 | 0.172 | 0.843 | 0.409 | 0.400 | 0.253 | 0.250 |
| Other² | 0.059 | 0.040 | 0.014 | 0.042 | 0.082 | 0.297 | 0.143 | 0.116 | 0.080 | 0.120 |
| Total | 5.661 | 5.573 | 4.513 | 4.700 | 2.614 | 2.363 | 3.773 | 3.737 | 4.013 | 4.153 |

¹Not detected.
²Includes C_{14:0}, C_{17:0}, and C_{≧20:0}

Experimental Procedure

Each experimental period consisted of 3 wk of which the first 16 d were used as an adjustment period. Cows were housed in individual tie stalls and were fed for ad libitum intake (allowing for 5 to 10% orts) once daily at 0800 h. At each feeding, diet ingredients were thoroughly mixed (Steiner Roll-A-Mix; Steiner Corp., Orrville, Ohio). Feed intakes and orts were recorded daily. Samples of corn silage, alfalfa haylage, and concentrates fed to each cow and orts from each cow were collected on d 17 to 20 of each period, composited, oven-dried at 65° C, and ground through a Wiley mill (1-mm screen; Arthur H. Thomas, Philadelphia, Pa.) for later analysis. Milk production was recorded at each milking (0100 and 1230 h). Morning and evening milkings were sampled during the last 3 d (six consecutive milkings) of each period. Cows were exercised each morning from 0600 to 0800 h. Body weight was recorded weekly at 100 h every Tuesday.

From d 11 to 21 of each period, Cr₂O₃ was mixed with the concentrate portion of the diet at a rate of approximately 20 g/d per cow based on previous DM intake to measure duodenal nutrient flow and to calculate total tract digestibility. Actual Cr₂O₃ intake was determined by chemical analysis of feeds and orts. Duodenal and fecal samples (250 ml) were collected every 4 or 6 h during d 19 to 21 such that a composite of 12 samples represented sampling every 2 h over a 24-h period. Duodenal samples were collected after discarding the first surge of digesta to help reduce bile contamination. Duodenal samples were frozen at −20° C., thawed, composited, and freeze-dried before being ground through a 1-nmm screen.

Ruminal fluid samples for VFA and pH were collected every 2 h over a 12-h duration on d 21 of each experimental period beginning just before the morning feeding. Samples were collected from five distinct locations: the anterior blind sac, dorsal blind sac, upper and lower forage mat, and liquid phase of the ventral sac using a stainless steel sampling probe (10 ml of ruminal fluid per location). Ruminal fluid pH was determined by glass electrode, acidified to pH<2 with 1 ml of 50% (vol/vol) H₂SO₄, and frozen for VFA analysis.

Ruminal samples for isolating bacteria were obtained on d 21 of each period at 0, 4, 8, and 12 h after the morning feeding. Whole ruminal contents, acquired from the anterior, dorsal and midventral regions of the rumen, were squeezed through four layers of cheesecloth. five hundred milliliters of the strained ruminal fluid were collected at each sampling, combined with 125 ml of 0.9% NaCl and 37% formaldehyde solution to preserve rumen bacteria, and refrigerated at 5% C until centrifugation to isolate bacteria. Remaining ruminal contents were returned to the cow. Composited samples were centrifuged at 500 (g for 15 min at 4% C to remove feed particles and protozoa. The supernatant was centrifuged at 20,000 (g for 20 min at 4% C, washed with 0.9% NaCl, recentrifuged, and rinsed with distilled water to isolate the bacteria from the suspension. The remaining pellet was frozen, freeze-dried, and ground through a 1-mm screen for later analysis.

Analytical Procedures

Composited samples of alfalfa haylage, corn silage, concentrates, orts, duodenal digesta, and feces were analyzed for DM (100° C. for 24 h), OM (2), and N by the micro-Kjeldahl procedure (2). Feed samples were additionally analyzed for NDF (29), ADF (10), Ca, P, Mg, K, and Na (Northeast DHIA Forage Analysis Laboratory, Ithaca, N.Y.). Long-chain FA compositions of feed, fecal, duodenal, and milk samples were quantified by GLC as described by Gaynor et al. (8), except that feed, fecal, and duodenal methyl esters were purified through a silicic acid column (Bio-Sil A 100-200 mesh; Bio-Rad Laboratories, Hercules, Calif.) to remove contaminants prior to GLC analysis. Individual milk samples were analyzed for fat, protein, and lactose by Environmental Systems Services (College Park, Md.) using infrared analysis (Foss Milkoscan 104AB; Foss Food Technology Corp., Eden Prairie, Minn.). Ruminal VFA concentrations were determined by gas chromatography (Sigma 300; Perkin-Elmer Corp., Norwalk, Conn.) using ethyl butyrate as an internal standard in a 2-m glass column packed with GP 15% SP-1220/1% H₃PO₄ on 100/120 Chromosorb® (Supelco Inc., Bellefonte, Pa.). Helium was the carrier gas and the injector, column, and detector temperatures were 200, 140, and 220° C., respectively.

Concentrates, orts, duodenal digesta, and feces were analyzed for Cr according to the procedure of Suzuki and Early (26). Duodenal and total tract flows of DM were calculated by dividing the amount of daily Cr intake by Cr concentration measured in duodenal and fecal samples. Nutrient flows were calculated by multiplying DM flow by the concentration of the nutrient in duodenal or fecal DM. Digestibilities, expressed as a percentage, were computed as the difference between nutrient intake and flows divided by intake. True ruminal DM digestibility was estimated by correcting duodenal DM flow for bacterial DM.

Bacterial pellet was analyzed for DM and N. Bacterial N flow to the duodenum was determined by purine analysis (32), as modified by Ushida et al. (28), of duodenal digesta and bacterial isolates. Ammonia N was measured by distillation of samples treated with MgO (2).

Statistical Analysis

All data except ruminal pH and VFA were analyzed as a 4×4 Latin square using PROC GLM of SAS (22). The statistical model included the effect of cow, period, forage level, buffer, and forage (buffer interaction. Time and time (treatment interactions were added to the model for ruminal pH and VFA and anayzed using PROC MIXED of SAS (21).

Results

Intakes And Digestibilities Of Nutrients

Dry matter and OM intakes were 2.7 and 2.5 kg/d higher (P<0.01), respectively, for cows fed LF diets (Table 13), and NDF intakes were 2.2 kg/d higher (P<0.001) for cows fed HF diets because of the increased forage in the diet. There was a forage (buffer interaction (P<0.09) for which the LF diet without buffer decreased apparent ruminal DM digestibility compared with other diets (11.4% vs. 28.1%). The trend for the effect of the LF diet without buffer on apparent ruminal OM digestibility was similar to that shown for ruminal DM digestibility. Apparent ruminal NDF digestibility was lower for cows fed LF diets (P<0.001), but greater for cows diets with added buffer (P<0.010). There was a forage (buffer interaction for DM (P<0.07) and OM (P<0.08) flows to the duodenum because of reduced ruminal DM and OM digestibility for cows fed the LF diet without buffer compared with cows fed the other dietary treatments. True ruminal DM and OM digestibilities corrected for bacterial DM and OM flow resulted in greater ruminal disappearance of DM (47.3% vs. 38.6%; P<0.05) and OM (52.5% vs. 43.3%; P<0.05) for cows fed the HF diets than cows fed the LF diets. Total tract digestibility of NDF was lower for cows fed the LF diets (P<0.007).

TABLE 13
Intake and digestibility of DM, OM, and NDF of cows fed diets
that contained high (HF) and low (LF) proportions of forage with
(B) or without buffer (NB).

| | | | | | | Effect¹ | | |
|---|---|---|---|---|---|---|---|---|
| | HF | | LF | | | F | B | F × B |
| | NB | B | NB | B | SEM² | P | | |
| DM | | | | | | | | |
| Intake, kg/d | 20.6 | 21.9 | 23.7 | 24.1 | 0.8 | 0.01 | 0.37 | 0.66 |
| Duodenal flow, kg/d | 14.8 | 15.1 | 21.3 | 17.9 | 0.8 | 0.001 | 0.11 | 0.07 |
| Apparent ruminal digestibility, % | 27.8 | 30.3 | 11.4 | 26.2 | 3.1 | 0.02 | 0.03 | 0.09 |
| True ruminal digestibility, % | 46.8 | 47.7 | 33.5 | 43.6 | 3.6 | 0.05 | 0.18 | 0.25 |
| Total tract digestibility, % | 67.7 | 67.9 | 61.5 | 65.2 | 2.6 | 0.14 | 0.49 | 0.53 |
| OM | | | | | | | | |
| Intake, kg/d | 19.1 | 19.9 | 21.9 | 22.0 | 0.7 | 0.01 | 0.55 | 0.60 |
| Duodenal flow, kg/d | 12.3 | 12.5 | 18.3 | 15.1 | 0.8 | 0.001 | 0.11 | 0.08 |
| Apparent ruminal digestibility, % | 35.2 | 36.8 | 18.0 | 31.9 | 3.3 | 0.02 | 0.06 | 0.11 |
| True ruminal digestibility, % | 52.3 | 52.6 | 38.5 | 48.0 | 3.7 | 0.05 | 0.17 | 0.26 |
| Total tract digestibility, % | 68.6 | 68.8 | 62.8 | 66.6 | 2.8 | 0.21 | 0.50 | 0.55 |
| NDF | | | | | | | | |
| Intake, kg/d | 7.3 | 7.6 | 5.2 | 5.3 | 0.2 | 0.001 | 0.29 | 0.60 |
| Duodenal flow, kg/d | 2.9 | 2.7 | 3.3 | 2.8 | 0.3 | 0.42 | 0.29 | 0.54 |
| Apparent ruminal digestibility, % | 60.7 | 63.2 | 37.6 | 48.0 | 3.3 | 0.001 | 0.10 | 0.28 |
| Total tract digestibility, % | 54.4 | 54.6 | 27.2 | 34.7 | 5.9 | 0.007 | 0.54 | 0.56 |

¹F = Forage proportion,
B = buffer addition, and
F × B = forage by buffer interaction.
²Four observations per treatment

Intake and duodenal flow of individual and total FA are presented in Table 14. As expected, cows fed LF diets consumed greater C_{16:0}, cis-C_{18:1}, C_{18:2}, total C₁₈ and total FA, but reduced C_{16:1} and C_{18:3}, reflecting changes in FA composition of LF diets in addition to changes in DMI. There were negligible amounts of trans-C_{18:1} FA in the diet, and total intakes were <1 g/d regardless of dietary treatment.

TABLE 14
Intake and flow of fatty acids (FA) to the duodenum of cows fed diets that contained
high (HF) and low (LF) proportions of forage with (B) or without buffer (NB).

| | | | | | | Effect¹ | | |
|---|---|---|---|---|---|---|---|---|
| | HF | | LF | | | F | B | F × B |
| | NB | B | NB | B | SEM² | P | | |
| Intake, g/d | | | | | | | | |
| C_{16:0} | 126 | 134 | 149 | 156 | 6 | 0.01 | 0.28 | 0.96 |
| C_{16:1} | 3.5 | 3.4 | 1.5 | 2.2 | 0.3 | 0.002 | 0.31 | 0.23 |
| C18:0 | 27 | 37 | 28 | 37 | 4 | 0.83 | 0.05 | 0.91 |
| trans-C_{18:1} | 0.34 | 0.36 | 0.13 | 0.16 | 0.12 | 0.14 | 0.86 | 0.99 |
| cis-C_{18:1} | 145 | 151 | 204 | 215 | 9 | 0.001 | 0.37 | 0.77 |
| C_{18:2} | 379 | 381 | 501 | 510 | 17 | 0.001 | 0.77 | 0.85 |
| C_{18:3} | 83 | 85 | 58 | 58 | 3 | 0.001 | 0.71 | 0.72 |
| Total C₁₈ | 634 | 654 | 791 | 820 | 30 | 0.002 | 0.44 | 0.88 |
| Total | 789 | 811 | 954 | 996 | 36 | 0.002 | 0.40 | 0.79 |
| Duodenal flow, g/d | | | | | | | | |
| C_{16:0} | 156 | 163 | 208 | 197 | 7 | 0.001 | 0.75 | 0.23 |
| C_{16:1} | 4.2 | 6.0 | 7.0 | 4.9 | 0.4 | 0.10 | 0.87 | 0.004 |
| C_{18:0} | 439 | 456 | 535 | 551 | 21 | 0.005 | 0.49 | 0.96 |
| trans-C_{18:1} | 61 | 57 | 120 | 66 | 10 | 0.01 | 0.02 | 0.04 |
| cis-C_{18:1} | 84 | 88 | 140 | 116 | 8 | 0.002 | 0.26 | 0.12 |
| C_{18:2} | 86 | 84 | 166 | 109 | 11 | 0.003 | 0.03 | 0.04 |
| C_{18:3} | 11.2 | 10.7 | 12.9 | 8.6 | 1.0 | 0.86 | 0.05 | 0.10 |
| Total C₁₈ | 682 | 695 | 975 | 850 | 35 | 0.001 | 0.16 | 0.09 |
| Total | 916 | 946 | 1288 | 1131 | 41 | 0.001 | 0.17 | 0.06 |
| Biohydrogenation of unsaturated | | | | | | | | |
| FA, % | | | | | | | | |
| cis-C_{18:1} | 46.4 | 44.7 | 44.9 | 48.1 | 2.2 | 0.67 | 0.75 | 0.31 |
| C_{18:2} | 79.0 | 79.3 | 73.3 | 79.3 | 1.2 | 0.05 | 0.03 | 0.05 |
| C_{18:3} | 87.8 | 88.1 | 81.9 | 85.9 | 1.0 | 0.006 | 0.07 | 0.11 |
| Total C₁₈³ | 72.4 | 72.1 | 66.4 | 71.2 | 1.2 | 0.03 | 0.11 | 0.80 |

¹F = Forage proportion, B = buffer addition, and F × B = forage by buffer interaction.
²Four observations per treatment.
³Includes cis-C_{18:1}, C_{18:2}, and C_{18:3.}

Flow of both individual and total FA to the duodenum was increased by the LF diets (Table 14) because of increased FA intake. Forage (buffer interactions existed for C_{16:1}, trans-C_{18:1} FA, C_{18:2}, C_{18:3} total C₁₈, and total FA because of elevated levels of FA flow in cows fed LF diets without buffer. Flow of C_{16:0} C_{18:0}, and cis-C_{18:1} increased for cows fed LF diets. Increased FA flow in cows fed the LF diet without buffer reflected not only increased FA intake, but also the low ruminal DM digestibility (Table 13) resulting from the LF diet without buffer.

The extent of biohydrogenation of unsaturated C₁₈ FA was reflected by changes in flow of C_{18:0} to the duodenum as a percentage of the total C₁₈ FA flow. Duodenal C_{18:0} flow was 55% of the total C₁₈ FA flow for cows fed the LF diet without buffer compared with 65% of C₁₈ FA flow for cows fed the other diets. The flow of trams-C_{18:1} FA was 120 g/d for cows fed the LF diet without buffer (P<0.04) compared with 57 to 66 g/d for cows fed the other diets as a result of less complete ruminal biohydrogenation. Flows of C_{18:2} (P<0.04) and C_{18:3} (P<0.10) were increased in cows fed the LF diet without buffer compared with flows of C_{18:2} and C_{18:3} in cows fed other diets, again indicating reduced biohydrogenation for unsaturated C₁₈ FA cows fed the LF diet without buffer.

Ruminal biohydrogenation of unsaturated C₁₈ FA in the diet can be estimated from changes in the duodenum and intake of C_{18:0}, C_{18:1}, C_{18:2}, and C_{18:3} of total C₁₈ FA (31).

Biohydrogenation (percentage)=100−[100×(UFA/TFA)/(IUFA/ITFA)] Where

UFA=duodenal flow of an individual unsaturated C₁₈ FA,

TFA=duodenal flow of total C₁₈ FA,

IUFA=intake of an individual unsaturated C₁₈ FA, and

ITFA=intake of total C₁₈ FA.

It should be noted that using this index of biohydrogenation, incomplete biohydrogenation of C_{18:3} and C_{18:2} would be reflected in lower apparent biohydrogenation C_{18:2} and C_{18:1}. Within C₁₈ FA, as the degree of unsaturation increased, the extent of biohydrogenation increased, regardless of dietary treatment. Biohydrogenation of C_{18:2}, C_{18:3}, and total C₁₈ FA was decreased in the LF diet without buffer compared with that in other dietary treatments. Biohydrogenation of cis-C_{18:1} was not affected by treatment (Table 14).

Fecal FA (grams/day) did not differ for C_{16:1}, C_{18:0}, C_{18:3}, total C₁₈ and total FA among treatments (Table 15). Fecal C_{16:0}, cis-C_{18:1}, and C_{18:2} flows were greater in cows fed LF diets which corresponded with greater duodenal flows of FA. There was a forage (buffer interaction (P<0.03) for fecal trans-C_{18:1} FA for which trans-C_{18:1} FA were higher for cows fed the LF diet without buffer than for cows fed other dietary treatments.

Apparent total tract digestibility of C_{16:0}, cis-C_{18:1}, total C₁₈, and total FA was not affected by forage or buffer treatment (Table 15). High concentrate diets decreased apparent digestibility of C_{18:2} and C_{18:3} compared with HF diets. This result was most likely due to reductions in ruminal biohydrogenation for these FA (Table 14). Apparent digestibility of C_{18:0} in cows fed diets without added buffer was lower than that in cows fed diets with added buffer. Because of hydrogenation of unsaturated C₁₈ FA in the rumen, apparent digestibility of C_{18:0} was negative for all diets, resulting from a large quantity of C_{18:0} passing through the feces in comparison with consumption of C_{18:0}. Apparent postruminal digestibilities of individual and total FA did not differ among treatments (data not presented).

TABLE 15
Focal fatty acids and total tract apparent digestibilities of fatty acids
by cows fed diets that contained high (HF) and low (LF) proportions
of forage with (B) or without buffer (NB).

| | | | | | | Effect¹ | | |
|---|---|---|---|---|---|---|---|---|
| | HF | | LF | | | F | B | F × B |
| | NB | B | NB | B | SEM² | P | | |
| Focal flow, g/d | | | | | | | | |
| C_{16:0} | 36 | 38 | 52 | 44 | 6 | 0.10 | 0.66 | 0.40 |
| C_{16:1} | 1.2 | 1.6 | 1.3 | 1.7 | 0.4 | 0.46 | 0.64 | 0.61 |
| C_{18:0} | 93 | 105 | 136 | 108 | 21 | 0.33 | 0.72 | 0.38 |
| trans-C_{18:1} | 9.1 | 11.9 | 18.8 | 10.1 | 2.1 | 0.10 | 0.20 | 0.03 |
| cis-C_{18:1} | 17 | 18 | 32 | 26 | 4 | 0.02 | 0.52 | 0.37 |
| C_{18:2} | 18 | 16 | 37 | 27 | 6 | 0.04 | 0.35 | 0.48 |
| C_{18:3} | 2.1 | 1.7 | 2.4 | 1.7 | 0.3 | 0.65 | 0.13 | 0.65 |
| Total C₁₈ | 139 | 153 | 227 | 173 | 30 | 0.12 | 0.53 | 0.30 |
| Total | 206 | 222 | 314 | 247 | 37 | 0.12 | 0.51 | 0.29 |
| Total tract digestibility, % | | | | | | | | |
| C_{16:0} | 71.7 | 71.3 | 66.5 | 71.6 | 2.8 | 0.41 | 0.43 | 0.36 |
| C_{16:1} | 63.4 | 68.8 | 1.1 | 7.7 | 28.0 | 0.07 | 0.84 | 0.98 |
| C_{18:0} | −250 | −193 | −371 | −190 | 44 | 0.23 | 0.03 | 0.21 |
| cis-C_{18:1} | 88.4 | 88.1 | 84.9 | 87.6 | 1.1 | 0.12 | 0.32 | 0.21 |
| C_{18:2} | 95.4 | 95.8 | 93.0 | 94.6 | 0.9 | 0.06 | 0.28 | 0.48 |
| C_{18:3} | 97.5 | 98.0 | 96.0 | 97.1 | 0.5 | 0.04 | 0.15 | 0.55 |
| Total C₁₈ | 78.0 | 76.5 | 72.3 | 79.0 | 3.2 | 0.65 | 0.45 | 0.25 |
| Total | 73.8 | 72.5 | 68.1 | 75.3 | 3.1 | 0.66 | 0.38 | 0.22 |

¹F = Forage proportion, B = buffer addition, and F × B = forage by buffer interaction.
²Four observations per treatment.

Ruminal pH And VFA

Ruminal pH and VFA concentons, averaged across sampling times, are shown in Table 16. Ruminal pH was lower in cows fed LF diets (P<0.02). Addition of buffer increased ruminal pH by 0.19 units for cows fed the LF diet compared with only 0.02 units for cows fed the HF diets (P<0.07). Rumial pH decreased with time after feeding for all treatments (P<0.001) (FIG. 1). With the exception of the 0-h sampling time, ruminal pH for cows fed the LF diet without buffer was lower than that for cows fed the other three diets. The addition of buffer to the LF diet resulted in a greater increase in ruminal pH than the addition of buffer to the HF diet.

TABLE 16
Ruminal pH and VFA concentrations of cows fed diets that contained high
(HF) and low (LF) proportions of forage with (B) or without buffer (NB).

| | | | | | | Effect¹ | | |
|---|---|---|---|---|---|---|---|---|
| | HF | | LF | | | F | B | F × B |
| | NB | B | NB | B | SEM² | P | | |
| pH | 6.13 | 6.15 | 5.83 | 6.02 | 0.04 | 0.002 | 0.04 | 0.07 |
| Total VFA, mM | 89.00 | 97.20 | 110.10 | 112.30 | 3.50 | 0.002 | 0.18 | 0.41 |
| VFA, mol/100 mol | | | | | | | | |
| Acetate | 64.2 | 62.2 | 58.0 | 58.5 | 1.2 | 0.006 | 0.57 | 0.34 |
| Proprionate | 20.5 | 22.7 | 26.5 | 24.4 | 1.3 | 0.02 | 0.98 | 0.15 |
| Isobutyrate | 0.74 | 0.80 | 0.54 | 0.69 | 0.11 | 0.21 | 0.36 | 0.67 |
| Butyrate | 10.8 | 10.6 | 11.6 | 12.6 | 0.6 | 0.04 | 0.51 | 0.30 |
| Isovalerate | 1.42 | 1.62 | 1.25 | 1.56 | 0.13 | 0.41 | 0.09 | 0.70 |
| Valerate | 1.72 | 1.70 | 1.75 | 1.85 | 0.16 | 0.62 | 0.80 | 0.74 |

¹F = Forage proportion, B = buffer addition, and F × B = forage by buffer interaction.
²Four cows per treatment; seven repeated measures.

As expected, total VFA concentration in the rumen increased in cows fed the LF diets (Table 16). The molar proportion of acetate was decreased (58.3% vs. 63.3%), propionate was increased (25.5% vs. 21.6%), and butyrate was increased (12.1% vs. 10.7%) in cows fed LF diets compared with proportions of those acids in cows fed HF diets Buffer addition did not affect VFA concentrations.

N Metabolism

Intake of N was higher for cows fed LF diets than for cows fed HF diets (731 vs. 635 g/d) because of higher DMI for cows fed LF diets (Table 17). Duodenal flows of total N (P<0.001), ammonia N (P<0.02), NAN (P<0.001), bacterial N (P<0.005), and nonammonia, nonbacterial N (P<0.008) were increased for cows fed the LF diets. Buffer addition decreased the flow of total N (P<0.005), ammonia N (P<0.03), and NAN (P<0.007). Also, there was a forage (buffer interaction for which total N (P<0.009), ammonia N (P<0.009, and NAN (P<0.02) were increased in cows fed LF diet without buffer compared with those in cows fed other dietary treatments. These changes most likely were due to increased DM flow for cows fed the LF diet without buffer because of the low ruminal digestibility of DM.

TABLE 17
Digestion of N and microbial flow to the small intestine of cows fed diets that
contain high (HF) and low (LF) proportions of forage with (B) or without (NB).

| | | | | | | Effect¹ | | |
|---|---|---|---|---|---|---|---|---|
| | HF | | LF | | | F | B | F × B |
| | NB | B | NB | B | SEM² | P | | |
| Intake, g/d | 634 | 635 | 717 | 745 | 24 | 0.007 | 0.58 | 0.60 |
| Duodenal flow, g/d | | | | | | | | |
| Total N | 674 | 666 | 930 | 797 | 16 | 0.001 | 0.005 | 0.009 |
| Ammonia N | 53 | 55 | 72 | 54 | 3 | 0.02 | 0.03 | 0.009 |
| NAN | 621 | 611 | 858 | 743 | 16 | 0.001 | 0.007 | 0.02 |
| Bacterial N | 300 | 295 | 425 | 351 | 21 | 0.005 | 0.11 | 0.16 |
| Nonammonia, nonbacterial N | 321 | 316 | 433 | 392 | 24 | 0.008 | 0.38 | 0.49 |
| Duodenal flow, g/100 g of N | | | | | | | | |
| Ammonia N | 7.7 | 8.3 | 7.7 | 6.8 | 0.4 | 0.06 | 0.64 | 0.07 |
| NAN | 92.3 | 91.7 | 92.3 | 93.2 | 0.4 | 0.06 | 0.64 | 0.07 |
| Bacterial N | 44.6 | 44.2 | 45.5 | 44.2 | 2.4 | 0.88 | 0.73 | 0.86 |
| Bacterial synthesis, g of N/kg of | 31.2 | 29.1 | 50.6 | 33.4 | 2.0 | 0.001 | 0.003 | 0.008 |
| OMTD³ | | | | | | | | |
| Focal N, g/d | 184 | 191 | 255 | 225 | 20 | 0.04 | 0.600 | 0.40 |
| Total tract digestibility, % | 71.0 | 69.9 | 64.9 | 69.3 | 2.1 | 0.17 | 0.46 | 0.25 |

¹F = Forage proportion, B = buffer addition, and F × B = forage by buffer interaction.
²Four observation per treatment.
³OM truly digested in the rumen.

Efficiency of bacterial protein synthesis (gram of bacterial N per kilogram of OM truly digested) was higher for cows fed the LF diet without buffer (P<0.008) compared with other diets. Increased forage (P<0.001) and buffer addition (P<0.003) reduced bacterial efficiency. Fecal N, primarily caused by increased N intake was higher for cows fed LF diets (P<0.04). Apparently digested N in the total tract tended to be lower for cows fed the LF diet without buffer.

Milk Production And Composition

Milk production and 3.5% FCM were not affected by treatment (Table 18). Milk fat percentage was reduced for cows fed the LF treatments compared with cows fed the HF treatments (3.67% vs. 4.16%; P<0.01). Buffer addition increased milk fat percentage (3.76% vs 4.071%; P<0.07). The increase in milk fat percentage caused by buffer was numerically greater for cows fed the LF diet than for cows fed the HF diet. Milk protein percentage tended to be higher for cows fed the LF diets (3.71% vs. 3.61%; P<0.11). The production of milk components was not affected by treatment.

TABLE 18
Body weight, milk production, and milk composition or cows fed diets that contained
high (HF) and low (LF) proportion of forage with (B) or without buffer (NB).

| | | | | | | Effect¹ | | |
|---|---|---|---|---|---|---|---|---|
| | HF | | LF | | | F | B | F × B |
| | NB | B | NB | B | SEM² | P | | |
| BW, kg | 645 | 642 | 643 | 643 | 4 | 0.90 | 0.74 | 0.70 |
| Milk, kg/d | 28.1 | 29.3 | 31.5 | 29.8 | 1.3 | 0.18 | 0.85 | 0.29 |
| 3.5% FCM, kg/d | 30.8 | 32.3 | 30.9 | 31.1 | 1.5 | 0.70 | 0.58 | 0.67 |
| Milk components, % | | | | | | | | |
| Fat | 4.09 | 4.22 | 3.42 | 3.91 | 0.14 | 0.01 | 0.07 | 0.25 |
| Protein | 3.63 | 3.59 | 3.74 | 3.68 | 0.05 | 0.11 | 0.38 | 0.92 |
| Lactose | 4.78 | 4.71 | 4.80 | 4.86 | 0.07 | 0.23 | 0.90 | 0.37 |
| Production, kg/d | | | | | | | | |
| Fat | 1.14 | 1.21 | 1.07 | 1.12 | 0.07 | 0.27 | 0.42 | 0.92 |
| Protein | 1.02 | 1.05 | 1.15 | 1.07 | 0.04 | 0.12 | 0.59 | 0.22 |
| Lactose | 1.34 | 1.37 | 1.49 | 1.44 | 0.06 | 0.13 | 0.90 | 0.55 |

¹F = Forage proportion, B = buffer addition, and F × B = forage by buffer interaction.
²Four observations per treatment.

Concentrations and the daily yield of individual FA in milk are shown in Table 19. There were small but significant effects of buffer and forage on C_{15:0}, C_{16:0}, C_{18:0}, C_{18:2}, and C_{18:3} concentrations in milk fat. The concentration of trans-C_{18:1} FA in milk was greater for cows fed the LF diet without buffer than for cows fed the other dietary treatments (5.8% vs. 3.0%; P<0.06). The yield of trans-C_{18:1} FA in milk was also greater for cows fed the LF diet without buffer than for cows fed the other diets (56 vs. 33 g/d; P<0.06).

TABLE 19
Milk fatty acid concentration and fatty acid production of cows fed
diets that contained high (HF) and low (LF) proportions of forage
with (B) or without buffer (NB).

| | HF | | LF | | | Effect¹ | | |
|---|---|---|---|---|---|---|---|---|
| | NB | B | NB | B | SEM² | F | B | F × B |
| | (g/100 g of fat) | | | | | P | | |
| C_{14:0} | 12.4 | 12.9 | 12.8 | 12.9 | 0.3 | 0.40 | 0.33 | 0.41 |
| C_{15:0} | 1.3 | 1.3 | 1.7 | 1.5 | 0.13 | 0.08 | 0.71 | 0.53 |
| C_{16:0} | 32.3 | 33.4 | 29.2 | 31.1 | 0.7 | 0.005 | 0.05 | 0.70 |
| C_{16:1} | 2.1 | 2.0 | 2.2 | 2.0 | 0.1 | 0.43 | 0.30 | 0.75 |
| C_{17:0} | 0.65 | 0.64 | 0.67 | 0.60 | 0.02 | 0.68 | 0.08 | 0.18 |
| C_{18:0} | 12.0 | 11.5 | 10.0 | 12.1 | 0.6 | 0.29 | 0.24 | 0.06 |
| trans-C_{18:1} | 3.1 | 2.9 | 5.8 | 2.9 | 0.6 | 0.07 | 0.05 | 0.06 |
| cis-C_{18:1} | 23.6 | 22.4 | 22.4 | 23.3 | 0.5 | 0.77 | 0.80 | 0.09 |
| C_{18:2}³ | 0.65 | 0.57 | 0.67 | 0.51 | 0.05 | 0.60 | 0.07 | 0.48 |
| C_{18:2(0-6)} | 4.1 | 3.9 | 5.2 | 4.6 | 0.2 | 0.003 | 0.06 | 0.21 |
| C_{18:3} | 0.70 | 0.71 | 0.61 | 0.49 | 0.03 | 0.004 | 0.14 | 0.11 |
| C_{20:4} | 0.21 | 0.25 | 0.30 | 0.23 | 0.04 | 0.41 | 0.68 | 0.24 |
| | (g/d) | | | | | | | |
| C_{14:0} | 128 | 154 | 128 | 144 | 11 | 0.95 | 0.18 | 0.49 |
| C_{15:0} | 13 | 16 | 18 | 17 | 1.7 | 0.10 | 0.55 | 0.34 |
| C_{16:0} | 336 | 399 | 315 | 352 | 33 | 0.35 | 0.18 | 0.70 |
| C_{16:1} | 21 | 24 | 23 | 23 | 1.5 | 0.70 | 0.50 | 0.40 |
| C_{17:0} | 6.9 | 7.6 | 7.2 | 6.8 | 0.6 | 0.75 | 0.85 | 0.42 |
| C_{18:0} | 127 | 137 | 106 | 135 | 15 | 0.47 | 0.23 | 0.56 |
| trans-C_{18:1} | 33 | 33 | 56 | 33 | 5 | 0.07 | 0.07 | 0.06 |
| cis-C_{18:1} | 248 | 265 | 234 | 261 | 23 | 0.71 | 0.38 | 0.83 |
| C_{18:2}³ | 6.7 | 6.6 | 6.8 | 5.6 | 0.8 | 0.62 | 0.44 | 0.49 |
| C_{18:2(0-6)} | 43 | 46 | 55 | 51 | 4 | 0.07 | 0.89 | 0.44 |
| C_{18:3} | 7.6 | 8.3 | 6.3 | 5.5 | 0.8 | 0.04 | 0.92 | 0.37 |
| C_{20:4} | 1.9 | 2.8 | 3.7 | 2.5 | 0.6 | 0.28 | 0.82 | 0.18 |

¹F = Forage proportion, B = buffer addition, and F × B = forage by buffer interaction.
²Four observations per treatment.
³Isomers.

From the above, high grain diets not only provide polyunsaturated FA required to form trans-C_{18:1} FA as a result of incomplete biohydrogenation, but also alter the ruminal environment, which favors incomplete biohydrogenation and production of trans-C_{18:1} FA. Low pH appears to be a factor that results in inhibition of the last step of FA biohydrogenation, the conversion of trans-C_{18:1} FA to stearic acid. Dietary buffers increase ruminal pH when cows are fed high grain diets. Consequently, trans-C_{18:1} FA flow to the duodenum is reduced in cows fed dietary buffers, and milk fat depression is alleviated.

### EXAMPLE 3

In the course of studies, the inventor group has learned that tFA production in the rumen is controlled by the rumen pH and the source of dietary fats and oils. In addition several studies in animals and in mammalian tissue culture lines showed there to be more 18:2 present than would have been predicted based on the diet or media composition used to culture the cells. One explanation for this is that the desaturase enzymes present in the tissues were producing the 18:2 fatty acids from the 18:1 fatty acids. (This is not the normal source of these 18:2 FA. Generally 18:2 FA are considered “essential” in the diets of mammalian cells) (Luke, Subdoh, Cliff, Kalponia).

Supporting Information For Example 3

In Atal, et.al, “Comparison of Body Weight and Adipose Tissue in Male C57 B1/6J Milk Fed Diets, With and Without Trans Fatty Acids on Lipid Accumulations in 3T3-LI Cells” LIPIDS, Vol. 28, No.12 (1992) (both references herein incorporated by reference) the ratio of 18:2 to 20:4 in lipids is altered when mice or adipose cells are ‘fed’ trans fatty acid containing diets. It is generally observed that the production of 18:2 and 20:4 are interfered with by consuming 18:1 trans acids. These results are seen below:

| | | Polar Lipid | | Non-Polar Lipid | |
|---|---|---|---|---|---|
| | | 18:2/20:4 ratio | | 18:20/20:4 ratio | |
| Study | Species | cis | trans | cis | trans |
| Atal | C57B1/6J | 1.9 | 1.8 | 41 | 91 |
| | Mouse | | | | |
| | Fat Pad | | | | |
| Panagrahi | 3T3 cells | 1.6 | 1.9 P < 0.05 | 8 | 12 P < 0.05 |
| | Preadiposites | | | | |
| | 3T3 | 1.7 | 2.5 P < 0.05 | 10 | 14 P < 0.05 |
| | Differen- | | | | |
| | tiating | | | | |
| Caughman | MLTC-1 | 0.13 | 0.27 | 2 | 5 |
| Leydig | Tumor Cells | | | | |
| | Differen- | | | | |
| | tiating | | | | |

The alteration in 18:2/20:4 ratios can be due to more 18:2 or less 20:4. There was no evidence that the 20:4 is decreased so one interpretation would be that the 18:2 is increased. This could be due to decreased elongation and desaturation to 20:4 (the standard explanation) due to the effects of tFA, or to increased amounts of 18:2. Since 18:2 can only come from the diet and those levels were the same, then an increased presence of 18:2 could be due to synthesis from dietary cis 12 18:1 which would be desaturated at the delta 9 position. Thus CLA's (9c, 11t CLAa) are formed from C18 delta 11t.

In a student thesis by Cliff Caughmnan, 1987, MS thesis, Trans Fatty acid metabolism in Murine Leydig Tumor Cells (MLTC-1). (herein incorporated by reference) one of the inventors noticed that there was more 18:2n-6 present in the experimental cells (grown on a trans fatty acid containing media) than in those grown in the control treatment. Even though there was less fat in the experimental media 3383 ug, compared to the control 3553 ug, there was more 18:2 present at the end of the growth period when the media and cells were considered. The control conditioned media+cells had 218 ug 18:2 while the experimental conditioned media+cells had 378 ug 18:2 present (a difference of 160 ug). Likewise there was more 20:4n-6 (arachidonic) present in the experimental vials (˜37 for control and ˜47 for experimental) which indicates that there was not an inhibition in conversion of 18:2 to 20:4. This evidence coupled with a recent publication by Salminen et. al., “Dietary Trans Fatty Acids Increase Conjugated Linoleic Acid Levels in Human Serum” J. Nutr. Biochem., 1998, Vol. 9, pg. 93-98 (February) herein incorporated by reference, i.e. subjects fed PHVO is confirmation of the in vivo conversion of 18:1's to 18:2's.

### EXAMPLE 4

In a recent unpublished study the inventive group reviewed isomer distribution under different diet conditions and obtained the following results. American Dairy Science Association 1997 @ Guelph, Ontario, Canada

Diet: HC=25% forage: 75% concentrate (corn etc)

LC=60% forage: 40% concentrate

Buffer=1.5% NaHCO₃ and 0.5% MgO

Sodium bicarbonate and magnesium oxide

Design: HC or LC diets with/without buffer 2×2 factorial 4×4 Latin Square.

Four multiparous rumen fistulated cows in mid lactation.

Results: Rumen pH lowered when fed HC diets.

Rumen ph increased with buffer ˜0.2 pH units

Milk fat decreased with HC diet (p=0.01).

Buffer addition increased milk fat from 3.3% to 4.0% in HC group.

Milk tFA decreased from 5.7 to 2.9% during HC trt with buffer (buffer×forage p=0.001).

Trans 18:1 delta 5,6,7,8,9 isomers were small components and did not change with diet trt.

t-10 isomer ranged between 11 and 25% and was highest with the HC diets.

t-10 and t-12 peaks showed a buffer×Forage interaction.

t-15 responded to buffer and forage independently.

t-11 was always the major peak (˜33%) and was lower in the presence of buffer.

Conclusions: Diet not only had an effect on the total tFA contained in milk but also on milk fat isomer distribution.

A preliminary/feasibility study of cow mammary enzyme activities.

Design: Feed HC ir LC diets.

Three cows fed each diet. Recover mammary tissue at slaughter.

Enzyme assays for Fatty Acid Synthase (FAS), Acetyl CoA Carboxylase (ACC) and Glucose-6-Phosphate Dehydrogenase (G-6-P dH)

### Ruminal pH And Milk Production Of Cows Fed Low Or High Concentrate Diets With Or Without Buffer

| | Low Concentrate | | High Concentrate | | P< | |
|---|---|---|---|---|---|---|
| Parameter | No Buffer | B | No Buffer | B | Conc | Buffer* |
| pH | 6.1 | 6.1 | 5.8 | 6.0 | 0.002 | 0.04 |
| Milk, kg/d | 28 | 29 | 32 | 30 | 0.08 | 0.85 |
| Fat % | 4.1 | 4.2 | 3.4 | 3.9 | 0.01 | 0.07 |
| 3.5% FCM, kg/d | 31 | 32 | 31 | 31 | 0.7 | 0.6 |
| % tFA in milk fat | 3.1 | 2.9 | 5.8 | 2.9 | 0.07 | 0.05 |
| tFA g/d | 33 | 33 | 56 | 33 | 0.07 | 0.07 |

*No significant interactions.

Results:

| | | | | % |
|---|---|---|---|---|
| | LC Diet | HC Diet | | Decrease |
| | Crude | Crude | % Decrease | Biopsy |
| Enzyme | Homogenate | Homogenate | Biopsy 1997 | 1997 |
| FAS | | | | 54% |
| u/ml homogenate | 0.81 | 0.56 | 69% | |
| u/mg tissue | 2.3 | 1.7 | 74% | |
| ACC | | | | 61% |
| u/ml homogenate | 0.79 | 0.5 | 67% | |
| u/mg tissue | 2.3 | 1.4 | 61% | |
| G-6-PdH | | | | nd |
| u/ml homogenate | 0.55 | 0.73 | 75% | |
| u/mg tissue | 0.03 | 0.04 | 75% | |

3. 1997 Biopsy Study

Design: 2×2 single reversal

12 multiparous cows in midlactation

Two week preliminary period fed CT diet

Two week experimental periods fed either CR or HCS diets

Biospsy taken at the end of each treatment period

Milk composites of the last three days of each trt were analyzed for FA composition, fat, protein, etc.

Assays for ACC, FAS and ACC mRNA abundance were run for each diet trt.

The results show that enzyme production involved in milk fat formation by the mammary gland is affected by diet. High concentrate diets reduce the amount of fat producing enzymes thus fewer short chain fatty acids are made and incorporated into this milk. High concentrated diets with oil added produced tFA. The tFA decrease the fat synthesizing enzymes in the mammary gland; at the same time, these tFA themselves are incorporated into the milk fat both as t18:1 and CLA tFA's.

Results:

| | | | | % |
|---|---|---|---|---|
| Parameter | CT Diet | HCS Diet | P< | Decrease |
| % Milk Fat | 3.0 ± 0.17 | 1.7 ± 0.17 | 0.0004 | 57% |
| % tFA in Milk Fat | 1.9 ± 1.18 | 15.6 ± 1.18 | 0.0001 | \hat{ }8x |
| FAS enzyme activity | 13.2 ± 0.62 | 7.5 ± 0.62 | 0.0001 | 57% |
| ACC enzyme activity | 9.7 ± 0.3 | 3.8 ± 0.3 | 0.0001 | 40% |
| ACC mRNA | | decreased | | |
| abundance | | | | |

Short chain fatty acids were also decreased in the milk fat containing high levels of trans fatty acids. This is consistent with the observed decreases in enzyme activities.

Milk Fat FA Sources

Acetate and BHB* made in rumen are used in gland

to make FA. TFA made in rumen from diet fat.

BHB=*Beta hydrorzy butyrate

Mammary Gland

Make all the short chain fatty acids 6--->14.

4 comes from BHB

16 comes from diet and synthesis

Almost all the 18's come from diet

All the 18:2, 18:3 come from diet

tFA come from diet via biohydrogenation

### Ingredient Composition Of Biopsy Diet

| | |
|---|---|
| CT | HC |

| | |
|---|---|
| INGREDIENT | % of DM |

| | | |
|---|---|---|
| Forage | 60 | 25 |
| Corn Silage | 45.2 | 25 |
| Alfalfa haylage | 14.8 | 0 |
| Concentrate | 40 | 75 |
| Ground corn | 22.8 | 52.2 |
| Soyplus | 1.6 | 0 |
| Soybean meal | 13.8 | 15.4 |
| Urea | 0 | 0.6 |
| Dicalcium Phosphate | 0.52 | 0.67 |
| Limestone | 0.32 | 0.9 |
| NaCl | 0.4 | 0.4 |
| KCl | 0.52 | 0 |
| MgO | 0.16 | 0.3 |
| Dynamate | 0.6 | 0 |
| Trace mineral & vitamin mix | 0.16 | 0.15 |
| Soy oil | 0 | 3.6 |

DIET: Control (CT) = 36% corn silage, 24% alfalfa haylage; 40% concentrate.
Oil (HC) = 70% concentrate; 25% corn silage + 5% soybean oil.

### Chemical Composition Of Biopsy Diets

| | |
|---|---|
| CT | HC |

| | |
|---|---|
| INGREDIENT | % of DM |

| | | |
|---|---|---|
| DM % | 55.2 | 74.8 |
| Crude Protein | 17.5 | 17.3 |
| UIP | 6.3 | 6.4 |
| Sol. Protein | 5.5 | 5.3 |
| ADF Fiber | 18.1 | 8.2 |
| NDF Fiber | 31.2 | 17.5 |
| NF CHO | 40.7 | 53.1 |
| Energy NE MCAL/lb | 0.73 | 0.89 |
| Fat | 3.0 | 7.2 |
| Calcium | 0.69 | 0.72 |
| Phosphorus | 0.44 | 0.43 |
| Sodium | 0.25 | 0.22 |
| Magnesium | 0.30 | 0.22 |
| Sulfur | 0.23 | 0.22 |
| Potassium | 1.3 | 1.11 |
| Chlorine | 0.44 | 0.60 |

Diet Control (CT) = 36% corn silage; 24% alfalfa haylage; 40% concentrate
Oil (HC) = 70% concentrate; 25% corn silage + 5% soybean oil

### Mouse Milk Fat Levels When Fed Different Diets

| | | |
|---|---|---|
| CIS | TRANS | P< |

| | |
|---|---|
| DIET | Fat¹ as Volume % |

| | | | |
|---|---|---|---|
| High Fat High *EFA | 35 | 26 | 10⁻⁶ |
| Fat (40 enl %) | | | |
| EFA (12 en %) | | | |
| tFA (0 or 10 en %) | | | |
| Low Fat High EFA | 36 | 27 | 10⁻⁶ |
| Fat (20 en %) | | | |
| EFA (6 en %) | | | |
| tFA (0 or 15 en %) | | | |
| High Fat Low EFA | 37 | 24 | 10⁻⁶ |
| Fat (20 en %) | | | |
| EFA (2 en %) | | | |
| tFA (0 or 7 en %) | | | |
| Low Fat Low EFA | 35 | 24 | 10⁻⁶ |
| Fat (20 en %) | | | |
| EFA (2 en %) | | | |
| tFA (0 or 7 en %) | | | |

¹Wt % = 0.06 ± 0.73 Vol %.
*EFA = Essential Fatty Acids

### Fatty Acid Composition Of Biopsy Diets

| | | | |
|---|---|---|---|
| CT | HC | CT | HC |

| | | |
|---|---|---|
| Fatty Acid | % FAME | % DM |

| | | | | |
|---|---|---|---|---|
| 16:0 | 17.1 | 14.4 | 0.5 | 1.03 |
| 18:0 | 3.0 | 3.2 | 0.9 | 0.23 |
| 18:1t | nd | nd | nd | nd |
| 18:1c | 16.2 | 19.5 | .49 | 1.40 |
| 18:2 | 45.6 | 53.7 | 1.4 | 3.9 |
| 18:3 | 4.4 | 3.4 | 0.1 | 0.24 |

### EXAMPLE 6

As noted above, the prevailing view is that the ingestion of C_{18:1} trans fatty acids leads to milk fat depression in a lactating animal. Thus ingestion of C_{18:1} may be responsible for lactation failure in mammals including humans. However, it is now determined that C_{18:1} t11 trans fatty acid ingestion does not reduce milk fat in the lactation products of mammals.

C57/B1/6J mice fed a control diet (10 wt % fat) were switched to one of four isomer diets at day 6 of lactation after milk collection. Approximately 25% of the dietary fat (5 caloric %) during the treatment period was replaced with one of four specific isomers of oleic acid, either cis-9; cis-11; trans-9 or trams-11, other diet components were identical. The mice consumed these diets ad libitum for four days and were milked again on day 10 of lactation. Milk and fecal fatty acid composition was analyzed as fatty acid methyl esters by GC. Specific isomers were identified by relative retention times. Milk fat percentage was determined by the creamatocrit method and differences between day 6 and day 10 fat percentages were evaluated by a paired t-test.

Mice fed the trans-9 diet had statistically lower (P<0.001) milk fat on day 10 than on day 6. Although day 10 milk fat for animals fed cis-11 and trans-11 diets was numerically lower, the result was not statistically different as compared to the control. Cis-9 fed animals had identical fat values on days 6 and 10 (P>0.8). Fatty acid consumptions of the milk fats confirmed the dietary treatments. Each of the test isomers appeared in the milk from mice fed the different diets. The cis-11 isomer was normally present at a level of 3% of the fat on day 6 when animals were fed control diet, and at 10% on day 10 when fed the cis-11 diet. Likewise, the trans isomers, normally present in milk at <0.1% of the fat were identified at 5.8% for trans-9 and 3% for trans-11 although the dietary level was 28% of diet fat. Fecal fat and fatty acid compositions change with diet changes and high levels of the trans isomer (˜65%) indicated lack of absorption of these geometric isomers compared to their cis counterparts (30%).

The inventors have also learned that ingestion of C₁₈ trans 11 fats do not reduce milk fat levels in cows.

Thus, C₁₈ trans 11, a CLA precursor may be used as a feed supplement for breast feeding.

### EXAMPLE 5:

In yet another example six cows were infused post rumen with vegetable oils formulated to have the same fatty acid composition of monounsaturates and polyunsaturates. Six other cows were given 18:1 trans as part of the mixture and another six cows were infused with only 18:1 cis. The milk fat composition of all eighteen cows was analyzed for selected fatty acids. As shown in table on page 38b, post infusion of 18:1 trans results in milk fat reduction, but large increases in trans C_{18:1} per 100 gram of milk fat.

The table in 38(c) indicates that infusions of fat will slightly decrease C₁₀-C₁₆ fatty acids, but supplying trans enhances fat reduction in C₁₀-C₁₆ range. The results also indicate production of 18:2 n-6 formation from trans and cis infused cows.

### Milk Parameters Of Cows After Abomasal Infusion With Cis Or Trans Fats

| | TREATMENT | | | P< | |
|---|---|---|---|---|---|
| PARAMETER | Control | CIS | TRANS | Fat¹ | Isomer¹ |
| Number of cows | 6 | 6 | 6 | | |
| Milk, kg/d | 32 | 35 | 34 | 0.01 | ns |
| Fat % | 3.9 | 4.1 | 3.2 | 0.05 | 0.001 |
| 3.5% FCM, kg/d | 33.4 | 37.5 | 31.6 | ns | 0.001 |
| trans-C_{18:1} g/100 g fat | 1.6 | 1.7 | 14.0 | 0.001 | 0.001 |

¹Orthogonal constrasts: Fat = no infusion versus infusion of CIS and TRANS fat mixtures: and Isomer = infusion of cis versus trans fats.

### Fatty Acid Composition Of Milk Fat From Cows Receiving Abomasal Infusions Of Cis Or Trans Fat Mixtures

| SELECTED FATTY | TREATMENT | | | P< | |
|---|---|---|---|---|---|
| ACIDS | Control | CIS | TRANS | Fat¹ | Isomer¹ |
| 10:0 | 3.1 | 2.7 | 2.0 | ns | 0.001 |
| 12:0 | 5.3 | 3.9 | 2.9 | ns | 0.001 |
| 14:0 | 14.2 | 10.8 | 9.7 | 0.001 | 0.001 |
| 16:0 | 36.1 | 25.7 | 22.5 | 0.001 | 0.001 |
| 18:0 | 10.1 | 10.8 | 11.3 | ns | 0.01 |
| 18:1 trans | 1.6 | 1.7 | 14.0 | 0.001 | 0.001 |
| 18:1 cis | 18.5 | 33.3 | 24.1 | 0.001 | 0.001 |
| 18:2 n-6 | 2.5 | 4.8 | 5.1 | 0.001 | 0.001 |
| odd chain (13 to 19) | 4.9 | 3.3 | 4.4 | 0.001 | 0.01 |

¹Othogonal contrasts: Fat = no infusion versus infusion of CIS and TRANS fat mixtures; and Isomer = infusion of cis versus trans fats.

mammals without fear of lactation failure. (Lactation failure is defined as a mother abandoning breast feeding of her infant because the child remains hungry after breast feeding. The infant simply is not getting enough calories).

These results also suggest that a mouse, fed defined diets and milked to determine milk compositions produced from the diets can be used as a model to predict cows milk composition when fed like diets or diets leading to similar fats presented to the cows intestinal tract.

### EXAMPLE 7

Mouse Milking

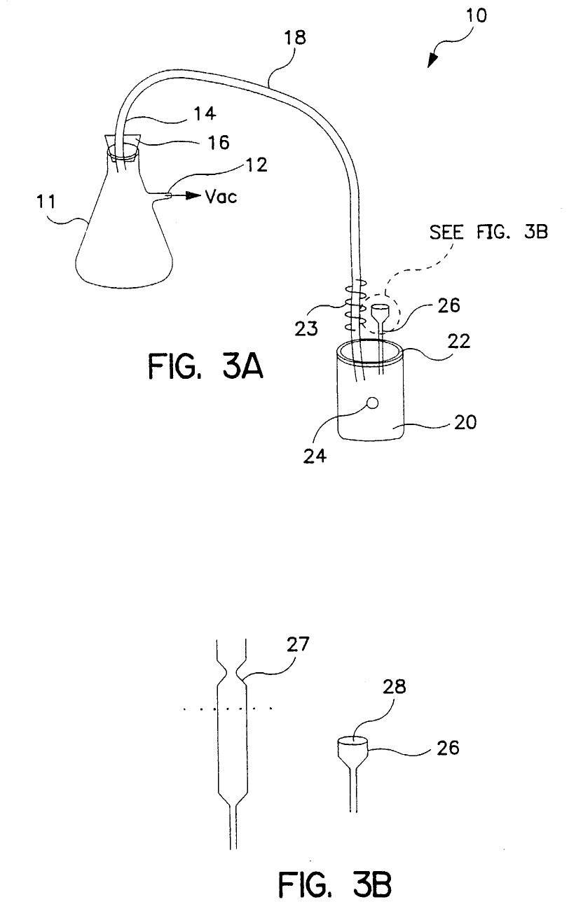

Shown in FIG. 3 is a device 10 used to milk a mouse. As shown, device 10 included a stoppered flask 11 connected through its arm 12 to a vacuum pump or aspirator. A glass tube 14 in fluid communication with the arm is inserted through stopper 16 and connected to hose 18 as shown. Hose 18 is approximately fifteen inches in length. A second component of the mouse milker includes a small closed cylinder 20 such as a pill dispenser obtained from a pharmacy and its cap 22. A single small diameter hole 24 (about 0.5 mm) is drilled into the wall of the cylinder. This hole is easily stopped by closing it with the tip of ones's index finger.

As shown in FIG. 3A, the second end of hose 18 is inserted through cap 22 of container 20. The end portion of hose 18 extending above the cap 22 is heavily taped with, for instance, surgical tape 23. This provides a “bullet” for the mouse to bite and prevents vacuum-line failure as the mouse cannot puncture hose 18.

A short glass tube 26, fashioned from a Pasture pipette 27 (inner diameter ¼ inch) is also inserted into the cap so that a first end extends into the hollow of the cap. The second end of the lube 26 is flame flared in the shape of a funnel (FIG. 3b) to receive a mouse teat.

A lactating mouse is milked after injection of oxytocin 0.3 ml immediately prior to milking. Milking is accomplished by creating a suction through tube 26 and inserting the funnel end 28 of tube 26 onto the teat of a lactating mouse in the upside-down position. As milk is collected into the funnel, a small capillary tube (i.e. the same as that used to collect human blood samples) is manually positioned within the funnel to collect milk by capillary action. Failure to collect the milk will expose the milk to evaporation thus increasing fat content of the sample.

Adjustments to the vacuum or amount of suction are made by lifting the index finger on and off of hole 24 on the cylinder. This on and off lifting of the finger also simulates the suckling pups and thus improves collection.

Although the invention is described with preferred embodiments, it is to be understood that variations and modifications may be used as will be appreciated by those of ordinary skill in the art. For instance, the invention includes feeding fats which contain tFA in the form of triglycenrdes, partial glycerides, free acids or as fatty acid derivatives such as salts, amides, esters, etc. These fatty acids or the substrate for the production of these fatty acids can be fed to various animals to produce the desired levels of CLA in the animal. Likewise human diets could be supplemented with the naturally occurring substrates or edible products of animals consuming CLA producing diets.

### REFERENCES FOR EXAMPLE 1

1. Association of Official Analytical Chemists. 1984. Official Methods of Analysis. 14th ed. AOAC, Washington, DC.

2. Astrup, H. N., L. Vik-Mo, A. Ekern, and F. Bakke. 1976. Feeding protected and unprotected oils to dairy cows. J. Dairy Sci. 59:426.

3. Banks, W., J, L. Clapperton, M. E. Kelly, A. G. Wilson, and R. J. M. Crawford. 1980. The yield, fatty acid composition and physical properties of milk fat obtained by feeding soya oil to dairy cows. J. Sci. Food Agric. 31:368.

4. Bickerstaffe, R., D. E. Noakes, and E. F. Annison. 1972. Quantitative aspects of fatty acid biohydrogenation. absorption and transfer into milk fat in the lactating goat, with special reference to the cis- and trans-isomers of octadecenoate and linoleate. Bichem. J. 130:607.

5. Casper, D. P., Schingoethe, D. J., Middaugh, R. P., and R. J. Baer. 1988. Lactational responses of dairy cows to diets containing regular and high oleic acid sunflower seeds. J. Dairy Sci. 71:1267.

6. Chouinard, P. Y., V. Girard, and G. J. Brisson. 1995. Influence of calcium salts of fatty acids (CSFA) with varying unsaturation on yield, composition, and fatty acid profile in Holstein milk. Can J. Anim. Sci. 75:656.(Abstr.)

7. Doreau, M., and A. Ferlay. 1994. Digestion and utilisation of fatty acids by ruminants. Anim. Feed Sci. Technol. 45:379.

8. Ferlay, A., J. Chabrot, Y. Elmeddah, and M. Doreau. 1993. Ruminal lipid balance and intestinal digestion by dairy cows fed calcium salts of rapeseed oil fatty acids or rapeseed oil. J. Aim. Sci. 71:2237.

9 Gaynor, P. J., R. A. Erdman, B. B. Teter, J. Sampugna, A. V. Capuco, D. R. Waldo, and M. Harnosh. 1994. Milk fat yield and composition during abomasal infusion of cis or trans octanodecenoates in holstein cows. J. Dairy Sci. 77:157.

10 Goering, H. K., and P. J. Van Soest. 1970. Forage Fiber Analyses (Apparatus, Reagents, Procedures, and Some Applications). Agric. Handbook No. 379. ARS-USDA, Washington, DC.

11 Harfoot, C. G. 1978. Lipid metabolism in the rumen. Prog. Lipid Res. 17:21.

12 Harfoot, C. G., and G. P. Hazlewood. 1988. Lipid metabolism in the rumen. Page 285 in The Rumen Microbial Ecosystem. P. N. Hobson, ed Elsevier Appl. Sci., London, England.

13 Kalscheur, K. F., R. A. Erdman, B. B. Teter, and L. S. Piperova. 1997. Effect of dietary forage level and buffer addition on duodenal flow of trans-C_{18:1} fatty acids and milk fat production in dairy cows. 80:(companion paper).

14 Katz, I., and M. Keeney. 1966. Characterization of the octadecenoic acids in rumen digesta and rumen bacteria. J. Dairy Sci. 49:962.

15 Klusmeyer, T. H., and J. H. Clark. 1991. Effects of dietary fat and protein on fatty acid flow to the duodenum and in milk produced by dairy cows. J. Dairy Sci. 74:3055.

16 National Research Council. 1989. Nutrient Requirements of Dairy Cattle. 6th rev. ed Natl. Acad. Sci., Washington, DC.

17 Noble, R. C. 1978. Digestion, absorption, and transport of lipids in ruminant animals. Prog. Lipid Res. 17:55.

18 Romo, G. A. 1995. Trans fatty acids: rumen in vitro production and their subsequent metabolic effects on energy metabolism and endocrine responses in the lactating dairy cow. Ph.D. Diss., Univ. Maryland, College Park.

19 Sampugna, J., L. A. Pallansch M. G. Enig, and M. Keeney. 1982. Rapid analysis of trans fatty acids on SP-2340 glass capillary columns. J., Chromotgr. 129:245.

20 SAS® Technical Report P-229. SAS/STAT® Software: Changes and Enhancements, Release 6.07. 1992. SAS Inst., Inc., Cary, N.C.

21 SAS® User's Guide: Statistics, Version 6.04 Edition. 1989. SAS inst., Inc., Cary, N.C.

22 Selner, D. R., and L. H. Schultz. 1980. Effects of feeding oleic acid or hydrogenated vegetable oils to lactating cows. J. Dairy Sci. 63:1235.

23 Suzuke, E. Y., and R. J. Early. 1991. Analysis of chromic oxide in small samples of feed and feces using chlorine bleach. Can. J. Anim. Sci. 66:157.

24 Ushida, K. B., B. Lassalas, and J. P. Jounay. 1985. Determination of assay parameters for RNA analysis in bacterial and duodenal samples by spectrophotometry. Influence of sample treatment and preservation. Reprod. Nutr. Dev. 25:1037.

25 Van Soest, P. J., J. B. Robertson, and B. A. Lewis. 1991. Methods for dietary fiber, neutral detergent fiber, and nonstarch polysaccharides in relation to animal nutrition. J. Dairy Sci. 74:3583.

26 Weisbjerg, M. R., C. F. Bursting, and T. Hvelplund. 1992. Fatty acid metabolism in the digestive tract of lactating cows fed tallow in increasing amounts at two feed levels. Acta Agric. Scand. Sect. A 42:106.

27 Wonsil, B. J., J. H. Herbein, and B. A. Watkins. 1994. Dietary and ruminally derived trans-18:1 fatty acids alter bovine milk lipids. J. Nutr. 124:556.

28 Wu, Z., O. A. Ohajuruka, and D. L. Palmquist. 1991. Ruminal synthesis, biohydrogenation, and digestibility of fatty acids by dairy cows. J. Dairy Sci. 4:3025.

29 Zinn, R. A., and F. N. Owens. 1986. A rapid procedure for purine measurement and its use for estimating net ruminal protein synthesis. Can. J. Arnim. Aci. 66:157.

### REFERENCES EXAMPLE 2

1 Askew, E. W., R. S. Emery, and J. W. Thomas. 1971. Fatty acid specificity of glyceride synthesis by homogenates of bovine mammary tissue. Lipids 6:777.

2 Association of Official Analytical Chemists. 1984. Official Methods of Analysis. 14th ed. AOAC, Washington, DC.

3 Davis, C. L., and R. E. Brown. 1970. Low-fat milk syndrome. Page 545 in Physiology of Digestion and Metabolism in the Ruminant. A. T. Phillipson, ed. Oriel Press, Newcastle Upon Tyne, England.

4 Doreau, M., and A. Ferlay. 1994. Digestion and utilization of fatty acids by ruminants. Anim. Feed Sci. Technol. 45:379.

5 Emery, R. S., and L. D. Brown. 1961. Effect of feeding sodium and potassium bicarbonate and potassium bicarbonate on milk fat, rumen pH, and volatile fatty acid production. J. Dairy Sci. 71:3246.

6 Erdman, R. A. 1988. Dietary buffering requirements of the lactating dairy cow. J. Dairy Sci. 71:3246.

7 Erdman, R. A., R. W. Hemken, and L. S. Bull. 1982. Dietary sodium bicarbonate and magnesium oxide for early postpartum lactating dairy cows: effects on production, acid-base metabolism, and digestion. J. Dairy Sci. 65:712.

8 Gaynor, P. J., R. A. Erdman, B. B. Teter, J. Sampugna, A. V. Capuco, D. R. Waldo, and M. Hamosh. 1994. Milk fat yield and composition during abosomal infusion of cis or trans octanodecenoates in Holstein cows. J. Dairy Sci. 77:157.

9 Gaynor. P. J., D. R. Waldo, A. V. Capuco, R. A. Erdman, L. W. Douglass, and B. B. Teter. 1995. Milk fat depression, the glucogenic theory, and trans-C_{18:1} fatty acids. J. Dair, Sci. 78:2008.

10 Goering, H. K., and P. J. Van Soest. 1970. Forage Fiber Analyses (Appartus, Reagents, Procedures, and Some Applications). Agric. Handbook No. 379. ARS-USDA, Washington, DC.

11 Grant, R. J., and D. R. Mertens. 1992. Influence of buffer pH and raw corn starch addition on in vitro fiber digestion kinetics. J. Dairy Sci. 75:2762.

12 Harfoot. C. G. 1978. Lipid metabolism in the rumen. Prog. Lipid Res. 17:21.

13 Harfoot, C. G. and G. P. Hazlewood. 1988. Lipid metabolism in the rumen. Page 285 in The Rumen Microbial Ecosystem. P. N. Hobson, ed. Elsevier Appl. Sci., London, England.

14 Harrison, D. G., and A. B. McAllan. 1980. Factors affecting microbial growth yields in the reticulo-rumen. Page 205 in Digestive Physiology and Metabolism in Ruminants. Y. Ruckebusch and P. Thivend, ed. AVI Publ. Co., Inc., Westport, Conn.

15 Klusmeyer, T. H., G. L. Lynch, J. H. Clark, and D. R. Nelson. 1991. Effects of calcium salts of fatty acids and proportion of forage in diet on ruminal fermentation and nutrient flow to duodenum of cows. J. Dairy Sci. 74:2220.

16 National Research Council. 1989. Nutrient Requirements of Dairy Cattle. 6th rev. ed. Natl. Acad. Sci., Washington, DC.

17 Noble, R. C. 1978. Digestion, absorption, and transport of lipids in ruminant animals. Prog. Lipid Res. 17:55.

18 Rode, L. M., D. C. Weaklv, and L. D. Satter. 1985. Effect of forage amount and particle size in diets of lactating dairy cows on site of digestion and microbial protein synthesis. Can. J. Anim. Sci. 68:445.

19 Rogers, J. A., C. L. Davis, and J. H. Clark. 1982. Alteration of rumen fermentation, milk fat synthesis and nutrient utilization with minerals salts in dairy cows. J. Dairy Sci. 65:577.

20 Romo, G. A. 1995. Trans fatty acids: rumen in vitro production and their subsequent metabolic effects on energy metabolism and endocrine responses in the lactating dairy cow. Ph.D. Diss, Univ. Maryland, College Park.

21 SAS® Technical Report P-229. SAS/STAT® Software: Changes and Enhancements, Release 6.07. 1992. SAS Inst., Inc., Cary, N.C.

22 SAS® User's Guide: Statistics, Version 6.04 Edition. 1989. SAS Inst., Cary, N.C.

23 Selner, D. R., and L. H. Schultz. 1980. Effects of feeding oleic acid or hydrogenated vegetable oils to lactating cows. J. Dairy Sci. 63:1235.

24 Stewart, C. S. 1977. Factors affecting the cellulolytic activity of rumen contents. Appl. Environ. Microbiol. 33:497.

25 Storry, J. E., and J. A. F. rook. 1965. The effect of a diet low in hay and high in flaked maize on milk fat secretion and on concentrations of certain constituents in the blood plasma of the cow. Br. J. Nutr. 19:101.

26 Suzuki, E. Y. and R. J. Early. 1991. Analysis of chromic oxide in small samples of feed and feces using chlorine bleach. Can J. Anim. Sci. 66:157.

27 Tamminina, S. 1981. Effect of the roughage/concentrate ratio on nitrogen entering the small intestine of dairy cows. Neth. J. Agric. Sci. 29:273.

28 Ushida, K. B., B. Lassalas, and J. P. Jounay. 1985. Determination of assay parameters for RNA analysis in bacterial and duodenal samples by spectrophotometry. Influence of sample treatment and preservation. Reprod. Nutr. Dev. 25:1037.

29 Van Soest, P. J., J. B. Robertson, and B. A. Lewis. 1991. Methods for dietary fiber, neutral detergent fiber, and nonstarch polysaccharide in relation to animal nutrition. J. Dairy Sci. 74:3583.

30 Wonsil, B. J., J. H. Herbein and B. A. Watkins. 1994. Dietary and ruminally derived trans-18:1 fatty acids alter bovine milk lipids. J. Nutr. 124:556.

31 Wu, Z., O. A. Ohajuruka, and D. L. Palmquist. 1991. Ruminal synthesis, biohydrogenation, and digestibility of fatty acids by dairy cows. J. Dairy Sci. 4:3025.

32 Zinn, R. A., and F. N. Owens. 1986. A rapid procedure for purine measurement and its use for estimating net ruminal protein synthesis. Can. J. Anim. Sci. 66:157.

What is claimed:

1. A method for decreasing the enzyme activity of Acetyl CoA Carboxylase (ACC) in ruminant production animals, comprising: feeding said animals high carbohydrate high PUFA diets which are not buffered.

2. A method for decreasing the enzyme activity of FAS in ruminant production animals, comprising, feeding said animals high carbohydrate high PUFA diets which are not buffered.

3. The method of claim 1 wherein the ruminant diet comprises ground corn in amounts up to about 75% by weight—a source of 18:2 n-6 and carbohydrates.

4. The method of claim 2 wherein the diet is supplemented with fats containing 18:2n-6.

5. A method for decreasing mRNA for acetyl CoA Carboxylase in production animals, comprising feeding said animals high carbohydrate diets with 18:2 or 18:3 FA or PHVO.

6. The method of claim 1 wherein the diet comprises ground corn in amounts up to 75% by weight.

7. The method of claims 3 wherein the diet is supplemented with oils comprising 18:3 n-3.

8. The method of claims 3 wherein the diet is supplemented with oils comprising other n-3 oils (fish oils work also).

9. A method of claim 7 wherein the cis trans conjugated acids in a mammal comprising, feeding to said mammal the food products of production animals fed diets high in carbohydrates and PUFA, said food products containing increased levels of 18:1 trans-11 acid as a result of consuming diets high in carbohydrates and PUFA or high in 18:1 t11.

10. The method of claim 7 wherein the cis trans conjugated acid is predominately 18:2 9C11t.

11. A feed composition or feed additive containing C₁₈ trans 11 fats in amounts effective to prevent lactation failure.

12. A model for predicting the composition of the fatty acid components or percentages in food products obtained from production animals, comprising a mouse fed a diet wherein the fatty acid components and/or percentages of the fatty acid in the diet are known, and the milk or meat product from the mouse are analyzed to predict the fatty acid components or percentages in the food products of the production animal fed diets similar to the diet fed the mouse.

13. The model of claim 12 wherein the mouse is C57/B1/6J.

14. A device for milking a mouse, comprising
a vacuum pump for generating suction;
a vacuum chamber;
a conduit connecting the pump to the vacuum chamber, a hollow tube a first end in vacuum communication with the vacuum chamber having a second funneled end for positioning over at least one teat of the mouse;
said vacuum chamber having a hole for disrupting the vacuum and for maintaining the vacuum by applying or removing a stopper for the hole.

15. The device of 14 wherein the conduit, at the vacuum chamber connecting end is wrapped with a device preventing a mouse being milked from puncturing the conduit.

16. A method for milking a mouse comprising
holding the mouse upside down;
exposing at least one teat of the mouse to the funneled end of the tube in the vacuum chamber of the device of claim 15;
contacting the teat with suction created by the vacuum pump;
collecting milk in the funneled end of the tube and withdrawing the milk from the tube before the milk enters the vacuum chamber by using a capillary pipette to capture the milk.
